SEQUENCE LISTING (25 sequences)

SEQ ID NO: 1 - DNA, 21 bp, Artificial Sequence
  crRNA portion
gatgatgtacatgacagcccg

SEQ ID NO: 2 - DNA, 22 bp, Artificial Sequence
  crRNA portion
gatcgctactctggcccaaagt

SEQ ID NO: 3 - DNA, 21 bp, Artificial Sequence
  crRNA portion
gatgtgacccacactttgggc

SEQ ID NO: 4 - DNA, 21 bp, Artificial Sequence
  crRNA portion
tcttctggagggctgtggtgg

SEQ ID NO: 5 - DNA, 21 bp, Artificial Sequence
  crRNA portion
accaatgctgatgaagacgaa

SEQ ID NO: 6 - DNA, 21 bp, Artificial Sequence
  crRNA portion
tagggaggaggtgatgcccga

SEQ ID NO: 7 - DNA, 21 bp, Artificial Sequence
  gRNA for use in targeting human AQP1
ctgagcatcgccacgctggcg

SEQ ID NO: 8 - DNA, 21 bp, Artificial Sequence
  gRNA for use in targeting human AQP1
accgatgctgatgaagacaaa

SEQ ID NO: 9 - DNA, 21 bp, Artificial Sequence
  gRNA for use in targeting human AQP1
ccgccgtctggttgttcccca

SEQ ID NO: 10 - DNA, 81 bp, Artificial Sequence
  tracrRNA component from an sgRNA recognised by SaCas9
gttttagtactctggaaacagaatctactaaaacaaggcaaaatgccgtgtttatctcgtcaacttgttggcgagattttt

SEQ ID NO: 11 - DNA, 130 bp, Artificial Sequence
  scAAV vectors
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 12 - DNA, 141 bp, Artificial Sequence
  scAAV vector
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 13 - protein, 736 aa, Artificial Sequence
  “native” ShH10 VP1 sequence
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNVQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQDQSGSAQNKDLLFSRGSPAGMSVQPKNWLPGPCYRQQRVSKTKTDNNNSNFTWTGASKYNLNGRESIINPGTAMASHKDDKNKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 14 - protein, 599 aa, Artificial Sequence
  VP2 native sequence
TAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNVQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQDQSGSAQNKDLLFSRGSPAGMSVQPKNWLPGPCYRQQRVSKTKTDNNNSNFTWTGASKYNLNGRESIINPGTAMASHKDDKNKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 15 - protein, 534 aa, Artificial Sequence
  VP3 native sequence
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNVQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQDQSGSAQNKDLLFSRGSPAGMSVQPKNWLPGPCYRQQRVSKTKTDNNNSNFTWTGASKYNLNGRESIINPGTAMASHKDDKNKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 16 - DNA, 21 bp, Artificial Sequence
  Mouse Aquaporin 1 Exon 1-1B
gatgatgtacatgacagcccg

SEQ ID NO: 17 - DNA, 21 bp, Artificial Sequence
  Mouse Aquaporin 1 Exon 1-1E
atcgctactctggcccaaagt

SEQ ID NO: 18 - DNA, 22 bp, Artificial Sequence
  Primer
ggaggaactgctggcatgcacc

SEQ ID NO: 19 - DNA, 22 bp, Artificial Sequence
  Primer
ctagagtgccagcctctgccct

SEQ ID NO: 20 - DNA, 7448 bp, Artificial Sequence
  AAV-SaCas9 plasmid mAqp1 exon 1 guide 1B
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtacctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatgatgtacatgacagcccggttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 21 - DNA, 7449 bp, Artificial Sequence
  AAV-SaCas9 plasmid mAqp1 exon 1 guide 1E
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtacctgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatcgctactctggcccaaagtgttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 22 - DNA, 510 bp, Artificial Sequence
  exon 1 of mouse Aqp1
aataggcccagcccaggctgtggctcagctcttggagggagttgagcaccaggcatccagcggttaagtcgaggtccctgccagcatggccagtgaaatcaagaagaagctcttctggagggctgtggtggctgagttcctggccatgaccctcttcgtcttcatcagcattggttctgccctaggcttcaattacccactggagagaaaccagacgctggtccaggacaacgtgaaggtgtcgctggcctttggtttgagcatcgctactctggcccaaagtgtgggtcacatcagcggtgctcacctcaaccctgcggtcacactggggctcctgctcagctgtcagatcagcatcctccgggctgtcatgtacatcatcgcccagtgtgtgggagccatcgtcgccacggccattctctcgggcatcacctcctccctagtcgacaattcacttggccgcaatgacgtgagtggggtatcttaggcttagggtgtctgtaggagaag

SEQ ID NO: 23 - DNA, 168 bp, Artificial Sequence
  genomic DNA alteratiosn sequenced from Mix sgRNA treated B6-RPE cells
aggtgtcgctggcctttggtttgagcatcgctactctggcccaaagtgtgggtcacatcagcggtgctcacctcaaccctgcggtcacactggggctcctgctcagctgtcagatcagcatcctccgggctgtcatgtacatcatcgcccagtgtgtgggagccatcg

SEQ ID NO: 24 - DNA, 84 bp, Artificial Sequence
  genomic DNA alteratiosn sequenced from Mix sgRNA treated B6-RPE cells
aggtgtcgctggcctttggtttgagcatcgctactctggcccaagctgtcatgtacatcatcgcccagtgtgtgggagccatcg

SEQ ID NO: 25 - DNA, 169 bp, Artificial Sequence
  genomic DNA alteratiosn sequenced from Mix sgRNA treated B6-RPE cells
aggtgtcgctggcctttggtttgagcatcgctactctggcccaaagtgtgggtcacatcagcggtgctcacctcaaccctgcggtcacactggggctcctgctcagctgtcagatcagcatcctccggcgctgtcatgtacatcatcgcccagtgtgtgggagccatcg

(19) United States
(12) Patent Application Publication
(10) Pub. No.: US 2021/0246453 A1
(43) Pub. Date: Aug. 12, 2021

(54) MATERIALS AND METHODS FOR MODULATING INTRAOCULAR AND INTRACRANIAL PRESSURE

(71) Applicant: The University of Bristol, Bristol Avon (GB)

(72) Inventor: Colin Jonathan CHU, Bristol Avon (GB)

(21) Appl. No.: 16/973,894

(22) PCT Filed: Jun. 11, 2019
(86) PCT No.: PCT/EP2019/065231

§ 371 Date: Dec. 10, 2020

(30) Foreign Application Priority Data
Dec. 6, 2018 (GB) 1809588.5

(51) Int. Cl. C12N 15/113; C12N 15/86

(52) U.S. Cl. CPC C12N 15/1138; C12N 15/1137; C12N 2750/14143; C12Y 402/01001; C12N 2310/20; C12N 15/86

(57) ABSTRACT

The invention relates to materials and methods for the modulation of intraocular and intracranial pressure, and treatment of associated conditions such as glaucoma and hydrocephalus. More specifically, the invention relates to adenoviral vectors of serotype ShH10, and their therapeutic use in transducing the CRISPR system into ciliary body or choroid plexus to modulate expression of aquaporin or carbonic anhydrase genes.

### CROSS-REFERENCE

This application is a 371 National Stage filing and claims the benefit under 35 U.S.C. § 120 to International Application No. PCT/EP2019/065231, filed Jun. 11, 2019, which claims priority to Great Britain Application No. 1809588.5, filed Jun. 12, 2018, each of which is incorporated herein by reference in its entirety.

### FIELD OF THE INVENTION

The invention relates to viral vectors of ShH10 serotype and their use for the modulation of intraocular and intracranial pressure, and treatment of associated conditions such as glaucoma and hydrocephalus.

### BACKGROUND TO THE INVENTION

Glaucoma is the leading cause of irreversible blindness worldwide and an estimated 11.2 million people will be bilaterally blind from the disease by 2020. Direct medical costs in the US alone are currently estimated at $3 billion per year, with 45% of the cost accounted for by prescription drug expenditure. The UK is comparable, where the prevalence of glaucoma is estimated at 2% of all people over the age of 40 years. Current treatment options are inadequate, exacerbated by the need for lifelong monitoring and therapy following diagnosis.

The initiating pathology is diverse and no definitive cure exists. However, clinical trials have shown that sufficient long-term reduction in intraocular pressure (IOP) largely prevents progression and visual loss by halting continued damage to the optic nerve.

Hydrocephalus or raised intracranial pressure is an important condition caused by a wide range of neurological disorders. The unifying cause is an imbalance between the production and drainage of cerebrospinal fluid (CSF). Many conditions such as congenital malformations, meningitis and brain tumours can cause hydrocephalus. Other conditions, such as idiopathic intracranial hypertension, can also lead to significant morbidity and blindness from elevated CSF pressure.

Current treatments involve oral medication with many unacceptable side-effects which limit their use. Surgical intervention in the form of shunting has a high rate of failure and complication (up to 48% over 5 years in children and 27% in adults in one study).

Thus there is a need for further treatment options for glaucoma, hydrocephalus and related conditions.

### SUMMARY OF THE INVENTION

The invention relates to adeno-associated virus (AAV) vectors which are able to transduce the ciliary body and choroid plexus in preference to surrounding tissues. In particular, it has been found that the ShH10 serotype of adeno-associated virus can provide effective and specific transduction of ciliary body and choroid plexus.

The ciliary body is involved in the production of aqueous humour in the eye, and has been targeted by a number of therapies for glaucoma, which aim to reduce intra-ocular pressure (IOP) by reducing aqueous humour production.

The choroid plexus is physiologically very similar to the ciliary body and is responsible for production of cerebrospinal fluid (CSF). An accumulation of CSF in the brain can lead to increased intracranial pressure (ICP) and conditions such as idiopathic intracranial hypertension and hydrocephalus.

Other types of virus, including other adenoviral serotypes, are either unable to transduce ciliary body and choroid plexus at all, or are insufficiently specific for these tissues to represent viable options for clinical use.

The viral vectors of the invention therefore enable a highly targeted gene editing approach to the treatment of conditions which may benefit from a reduction in intraocular or intracranial pressure, and/or a reduction in production of aqueous humour or CSF, such as glaucoma and hydrocephalus.

The invention provides an AAV vector virion of serotype ShH10, comprising:

(i) a nucleic acid sequence encoding an RNA-guided endonuclease; and
(ii) a nucleic acid sequence encoding a guide RNA complementary to a target sequence from an aquaporin gene or a carbonic anhydrase gene and capable of directing said RNA-guided endonuclease to said target sequence.

The invention also provides an AAV vector virion for use in a method of modulating intraocular pressure or production of aqueous humour, wherein the AAV vector is of serotype ShH10 and comprises:

(i) a nucleic acid sequence encoding an RNA-guided endonuclease; and
(ii) a nucleic acid sequence encoding a guide RNA complementary to a target sequence from an aquaporin gene or a carbonic anhydrase gene and capable of directing said RNA-guided endonuclease to said target sequence.

The invention also provides an AAV vector virion for use in a method of modulating intracranial pressure or production of CSF, wherein the AAV vector is of serotype ShH10 and comprises:

(i) a nucleic acid sequence encoding an RNA-guided endonuclease; and
(ii) a nucleic acid sequence encoding a guide RNA complementary to a target sequence from an aquaporin gene or a carbonic anhydrase gene and capable of directing said RNA-guided endonuclease to said target sequence.

The invention also provides the use of an AAV vector virion in the preparation of a medicament for modulating intraocular pressure or production of aqueous humour, wherein the AAV vector is of serotype ShH10 and comprises:

(i) a nucleic acid sequence encoding an RNA-guided endonuclease; and
(ii) a nucleic acid sequence encoding a guide RNA complementary to a target sequence from an aquaporin gene or a carbonic anhydrase gene and capable of directing said RNA-guided endonuclease to said target sequence.

The invention also provides the use of an AAV vector virion in the preparation of a medicament for modulating intracranial pressure or production of CSF, wherein the AAV vector is of serotype ShH10 and comprises:

(i) a nucleic acid sequence encoding an RNA-guided endonuclease; and
(ii) a nucleic acid sequence encoding a guide RNA complementary to a target sequence from an aquaporin gene or a carbonic anhydrase gene and capable of directing said RNA-guided endonuclease to said target sequence.

The invention also provides a method of modulating intraocular pressure or production of aqueous humour comprising administering an AAV vector virion to a subject, wherein the AAV vector is of serotype ShH10 and comprises:

(i) a nucleic acid sequence encoding an RNA-guided endonuclease; and
(ii) a nucleic acid sequence encoding a guide RNA complementary to a target sequence from an aquaporin gene or a carbonic anhydrase gene and capable of directing said RNA-guided endonuclease to said target sequence.

The invention also provides a method of modulating intracranial pressure or production of CSF comprising administering an AAV vector virion to a subject, wherein the AAV vector is of serotype ShH10 and comprises:

(i) a nucleic acid sequence encoding an RNA-guided endonuclease; and
(ii) a nucleic acid sequence encoding a guide RNA complementary to a target sequence from an aquaporin gene or a carbonic anhydrase gene and capable of directing said RNA-guided endonuclease to said target sequence.

The vectors and methods of the invention are thus useful in the treatment of conditions or symptoms which can benefit from, or be alleviated by, modulation of intraocular pressure, intracranial pressure, production of aqueous humour, or production of CSF. Treatment may be therapeutic (for a pre-existing condition or symptom) or prophylactic (seeking to prevent, inhibit or delay development of a condition or symptom in an individual at risk thereof). “Modulation” in the context of the present invention typically indicates a reduction in the relevant characteristic, but may also indicate a tendency to inhibit an increase. Thus modulation may constitute an absolute reduction in the relevant characteristic, but may also constitute maintaining a steady state of the relevant characteristic, or providing a slower rate of increase than would otherwise occur in the absence of treatment.

Elevated intraocular pressure (ocular hypertension) is a major risk factor in the development of glaucoma. A reduction of intraocular pressure (IOP) has been shown to be beneficial, even in types of glaucoma where IOP is within the normal range. Thus the vectors and methods of the invention are useful in the treatment of ocular hypertension and/or glaucoma.

The glaucoma may be primary or secondary.

Primary glaucoma may be open-angle glaucoma, closed-angle glaucoma or normal tension glaucoma (NTG).

Elevated intracranial pressure, excess production of CSF, or impaired CSF drainage may lead to conditions such as hydrocephalus and idiopathic intracranial hypertension. Thus the vectors and methods of the invention are useful in the treatment of hydrocephalus and idiopathic intracranial hypertension.

The hydrocephalus may be communicating hydrocephalus, including normal pressure hydrocephalus, or non-communicating hydrocephalus. In either case, it may be congenital or acquired.

An AAV vector virion contains a single stranded DNA genome, comprising “payload” sequence flanked by inverted terminal repeats (ITRs). Thus, when this specification refers to a vector virion comprising a particular nucleic acid sequence, it will be understood that the vector virion contains a single stranded DNA genome molecule comprising such a sequence. When a vector virion is said to contain two or more particular nucleic acid sequences, those sequences will typically form part of the same single stranded DNA genome molecule. Similarly, when a vector virion is said to encode a particular molecule (e.g. an RNA or protein), it will be understood that the vector virion contains a single stranded DNA genome comprising a sequence encoding that molecule.

The RNA-guided endonuclease may be a Cas9 enzyme, such as Staphylococcus aureus (SaCas9), Streptococcus pyogenes (SpCas9), Neisseria meningitidis (NM Cas9), Streptococcus thermophilus (ST Cas9), Treponema denticola (TD Cas9), or variants thereof such as SpCas9 D1135E, SpCas9 VRER, SpCas9 EQR or SpCas9 VQR.

Due to its relatively smaller size, SaCas9 and variants thereof may be preferred, in view of the limited coding capacity of an AAV genome.

Other RNA-guided endonucleases may also be useful, however, such as Cpf1.

The RNA-guided endonuclease is typically catalytically active. However, in some circumstances, a catalytically dead RNA-guided endonuclease may be employed. A catalytically dead RNA-guided endonuclease may also comprise a transcriptional repressor domain, such as a Kruppel associated box (KRAB) domain, CS domain, WRPW domain, MXI1, mSin3 interacting domain, or histone demethylase LSD1 domain. Thus the vector genome will contain a gene encoding a fusion protein comprising a catalytically dead RNA-guided endonuclease and a repressor domain.

The RNA-guided endonuclease may further comprise a nuclear localisation sequence effective in mammalian cells.

The guide RNA will typically be a sgRNA, especially when the endonuclease is a Cas9 enzyme.

However, the guide RNA may alternatively be a crRNA. In such circumstances, if required for endonuclease activity, e.g. when the endonuclease is a Cas9 enzyme, the vector virion may also comprise a nucleic acid sequence encoding a tracrRNA. Cpf1 is believed not to require a tracrRNA.

The aquaporin gene may be any aquaporin (AQP) gene whose product is expressed in the ciliary body or choroid plexus, such as AQP1, AQP2, AQP3, AQP4, AQP5, AQP6, AQP7 or AQP11.

In the ciliary body, AQP1, AQP4 and AQP5 are the most highly expressed aquaporins and may represent particularly good targets for ocular conditions.

AQP1 and AQP4 are also expressed in the choroid plexus, so may be particularly good targets for cerebral conditions.

In some embodiments, the aquaporin gene is AQP1 and the target sequence is located within exon 1. For example, the guide RNA may comprise a crRNA portion comprising or consisting of the sequence GATGATGTACATGACAGCCCG, GATCGCTACTCTGGCCCAAAGT, GATGTGACCCACACTTTGGGC, TCTTCTGGAGGGCTGTGGTGG, ACCAATGCTGATGAAGACGAA or TAGGGAGGAGGTGATGCCCGA.

For example, the guide RNA may comprise a crRNA portion comprising or consisting of the sequence GATGATGTACATGACAGCCCG or GATCGCTACTCTGGCCCAAAGT.

In aspects of the invention which involve delivery of two guide RNAs targeted to the AQP1 gene, any two of these crRNA sequences may be used, such as the sequences GATGATGTACATGACAGCCCG and GATCGCTACTCTGGCCCAAAGT.

The crRNA sequences shown above were designed for complementarity to murine AQP1 but could be expected to work in other mammalian species such as humans. The skilled person will be capable of designing suitable sequences for targeting any gene of choice in any given species. For example, a gRNA for use in targeting human AQP1 may comprise or consist of the sequence CTGAGCATCGCCACGCTGGCG, ACCGATGCTGATGAAGACAAA or CCGCCGTCTGGTTGTTCCCCA.

Similarly, the carbonic anhydrase (CAR) gene may be any CAR gene whose product is expressed in the ciliary body or choroid plexus, such as CAR2, CAR3, CAR4, CR5b, CARE, CAR8, CAR9, CAR10, CAR12 or CAR14. CAR15 is also expressed in the ciliary body in mouse. CAR2, CAR3, CAR4, CAR12 and CAR14, e.g. CAR2, CAR3 and CAR14, may represent particularly good targets in the eye. CAR2, CAR4 and CAR12 are believed to be highly expressed in choroid plexus.

The invention further provides a pharmaceutical composition comprising a vector virion as described, in combination with pharmaceutically acceptable carrier.

The pharmaceutical composition may be formulated for intraocular injection, and more particularly for intravitreal or intracameral injection (e.g. for ocular applications such as the treatment of glaucoma). The pharmaceutical composition may be formulated for central administration, i.e. administration direct to the central nervous system (CNS). Compositions for central administration may, for example, be formulated for intrathecal injection, or intracranial injection or infusion, e.g. by intracerebroventricular injection or infusion. Such administration will be particularly appropriate for treatment of hydrocephalus.

The invention further provides a packaging cell, producing an AAV vector virion as described.

The invention also provides a therapeutic kit comprising a plurality of populations of vector virions as described, wherein each population encodes a different guide RNA. The guide RNAs encoded by the different populations may be directed to target sequences within the same gene or within different genes. It may be particularly desirable to provide at least two populations of vector virions encoding different guide RNAs directed to the same gene, since this may increase efficiency of gene inactivation, e.g. by deleting a portion of the gene. Typically this approach requires catalytically active endonuclease.

Thus the kit may comprise first and second AAV vector virions (or populations of vector virions) as described, said first and second vectors (or populations of vector virions) encoding respective different first and second guide RNAs complementary to respective different first and second target sequences, wherein the first and second target sequences may be from the same aquaporin or carbonic anhydrase gene.

The different vector virions or populations of vector virions may be otherwise identical apart from the guide RNAs which they encode.

The different vector virions, or populations of vector virions, may be provided as part of the same composition or in separate compositions. Each composition may independently be a pharmaceutical composition comprising the respective vector virion, or population of virions, in combination with a pharmaceutically acceptable carrier.

Thus the invention provides a therapeutic kit comprising:

(a) a first AAV vector virion of serotype ShH10, comprising:
(i) a nucleic acid sequence encoding an RNA-guided endonuclease; and
(ii) a nucleic acid sequence encoding a first guide RNA complementary to a first target sequence from an aquaporin gene or a carbonic anhydrase gene and capable of directing said RNA-guided endonuclease to first said target sequence; and
(b) a second AAV vector virion of serotype ShH10, comprising:
(i) a nucleic acid sequence encoding an RNA-guided endonuclease; and
(ii) a nucleic acid sequence encoding a second guide RNA complementary to a second target sequence from an aquaporin gene or a carbonic anhydrase gene and capable of directing said RNA-guided endonuclease to said second target sequence.

The invention also provides an AAV vector virion for use in a method of modulating intraocular pressure or production of aqueous humour, wherein the AAV vector virion is of serotype ShH10 and comprises:

(i) a nucleic acid sequence encoding an RNA-guided endonuclease; and
(ii) a nucleic acid sequence encoding a first guide RNA complementary to a first target sequence from an aquaporin gene or a carbonic anhydrase gene and capable of directing said RNA-guided endonuclease to first said target sequence;
wherein the AAV vector virion is for administration in combination with a second AAV vector virion of serotype ShH10, comprising:
(i) a nucleic acid sequence encoding an RNA-guided endonuclease; and
(ii) a nucleic acid sequence encoding a second guide RNA complementary to a second target sequence from an aquaporin gene or a carbonic anhydrase gene and capable of directing said RNA-guided endonuclease to said second target sequence.

The invention also provides an AAV vector virion for use in a method of modulating intracranial pressure or production of CSF, wherein the AAV vector virion is of serotype ShH10 and comprises:

(i) a nucleic acid sequence encoding an RNA-guided endonuclease; and
(ii) a nucleic acid sequence encoding a first guide RNA complementary to a first target sequence from an aquaporin gene or a carbonic anhydrase gene and capable of directing said RNA-guided endonuclease to first said target sequence;
wherein the AAV vector virion is for administration in combination with a second AAV vector virion of serotype ShH10, comprising:
(i) a nucleic acid sequence encoding an RNA-guided endonuclease; and
(ii) a nucleic acid sequence encoding a second guide RNA complementary to a second target sequence from an aquaporin gene or a carbonic anhydrase gene and capable of directing said RNA-guided endonuclease to said second target sequence.

The invention also provides the use of an AAV vector virion in the preparation of a medicament for modulating intraocular pressure or production of aqueous humour, wherein the AAV vector virion is of serotype ShH10 and comprises:

(i) a nucleic acid sequence encoding an RNA-guided endonuclease; and
(ii) a nucleic acid sequence encoding a first guide RNA complementary to a first target sequence from an aquaporin gene or a carbonic anhydrase gene and capable of directing said RNA-guided endonuclease to first said target sequence;
wherein the AAV vector virion is for administration in combination with a second AAV vector virion of serotype ShH10, comprising:
(i) a nucleic acid sequence encoding an RNA-guided endonuclease; and
(ii) a nucleic acid sequence encoding a second guide RNA complementary to a second target sequence from an aquaporin gene or a carbonic anhydrase gene and capable of directing said RNA-guided endonuclease to said second target sequence.

The invention also provides the use of an AAV vector virion in the preparation of a medicament for modulating intracranial pressure or production of CSF, wherein the AAV vector virion is of serotype ShH10 and comprises:

(i) a nucleic acid sequence encoding an RNA-guided endonuclease; and
(ii) a nucleic acid sequence encoding a first guide RNA complementary to a first target sequence from an aquaporin gene or a carbonic anhydrase gene and capable of directing said RNA-guided endonuclease to first said target sequence;
wherein the AAV vector virion is for administration in combination with a second AAV vector virion of serotype ShH10, comprising:
(i) a nucleic acid sequence encoding an RNA-guided endonuclease; and
(ii) a nucleic acid sequence encoding a second guide RNA complementary to a second target sequence from an aquaporin gene or a carbonic anhydrase gene and capable of directing said RNA-guided endonuclease to said second target sequence.

The invention also provides a method of modulating intraocular pressure or production of aqueous humour comprising administering first and second AAV vector virions to a subject, wherein the first AAV vector virion is of serotype ShH10 and comprises:

(i) a nucleic acid sequence encoding an RNA-guided endonuclease; and
(ii) a nucleic acid sequence encoding a first guide RNA complementary to a first target sequence from an aquaporin gene or a carbonic anhydrase gene and capable of directing said RNA-guided endonuclease to first said target sequence;
and the second AAV vector virion is of serotype ShH10 and comprises:
(i) a nucleic acid sequence encoding an RNA-guided endonuclease; and
(ii) a nucleic acid sequence encoding a second guide RNA complementary to a second target sequence from an aquaporin gene or a carbonic anhydrase gene and capable of directing said RNA-guided endonuclease to said second target sequence.

The invention also provides a method of modulating intracranial pressure or production of CSF comprising administering first and second AAV vector virions to a subject, wherein the first AAV vector virion is of serotype ShH10 and comprises:

(i) a nucleic acid sequence encoding an RNA-guided endonuclease; and
(ii) a nucleic acid sequence encoding a first guide RNA complementary to a first target sequence from an aquaporin gene or a carbonic anhydrase gene and capable of directing said RNA-guided endonuclease to first said target sequence;
and the second AAV vector virion is of serotype ShH10 and comprises:
(i) a nucleic acid sequence encoding an RNA-guided endonuclease; and
(ii) a nucleic acid sequence encoding a second guide RNA complementary to a second target sequence from an aquaporin gene or a carbonic anhydrase gene and capable of directing said RNA-guided endonuclease to said second target sequence.

In all such cases, the first and second target sequences are different, and may be from the same gene or different genes. As described above, it may be desirable for the first and second target sequences to be from the same gene, since this may increase efficiency of gene inactivation, e.g. by deleting a portion of the gene.

It is also possible to deliver an RNA-guided endonuclease and a guide RNA by co-administration of separate vectors.

Thus the invention also provides a therapeutic kit comprising:

(a) a first AAV vector virion of serotype ShH10, comprising a nucleic acid sequence encoding an RNA-guided endonuclease;
and
(b) a second AAV vector virion of serotype ShH10, comprising a nucleic acid sequence encoding a guide RNA complementary to a target sequence from an aquaporin gene or a carbonic anhydrase gene and capable of directing said RNA-guided endonuclease to said target sequence.

The second vector virion may encode a plurality of guide RNAs, e.g. two guide RNAs, each complementary to a different target sequence. The target sequences may be from the same gene or different genes. As described above, it may be desirable for two target sequences to be from the same gene, since this may increase efficiency of gene inactivation, e.g. by deleting a portion of the gene.

Each guide RNA may be an sgRNA or a crRNA.

Where the vector encodes a plurality of sgRNAs, the sgRNAs may each comprise an identical crRNA component.

Where the vector encodes a crRNA, or a plurality of crRNAs, it may also encode a compatible tracrRNA, if required for activity of the endonuclease encoded by the first vector. However, any tracrRNA may, additionally or alternatively, be encoded by the first vector.

The first and second vector virions may be provided as part of the same composition or in separate compositions. Each composition may independently be a pharmaceutical composition comprising the respective vector virion or virions in combination with a pharmaceutically acceptable carrier.

The invention also provides an AAV vector virion for use in a method of modulating intraocular pressure or production of aqueous humour, wherein said vector virion is of serotype ShH10 and comprises a nucleic acid sequence encoding an RNA-guided endonuclease, and is for administration in conjunction with a second AAV vector virion of serotype ShH10, said second vector virion comprising a nucleic acid sequence encoding a guide RNA complementary to a target sequence from an aquaporin gene or a carbonic anhydrase gene and capable of directing said RNA-guided endonuclease to said target sequence.

The invention further provides an AAV vector virion for use in a method of modulating intraocular pressure or production of aqueous humour, wherein said vector virion is of serotype ShH10 and comprises a nucleic acid sequence encoding a guide RNA complementary to a target sequence from an aquaporin gene or a carbonic anhydrase gene and is capable of directing an RNA-guided endonuclease to said target sequence; and is for administration in conjunction with a second AAV vector virion of serotype ShH10, said second vector virion comprising a nucleic acid sequence encoding said RNA-guided endonuclease.

The invention also provides an AAV vector virion for use in a method of modulating intracranial pressure or production of CSF, wherein said vector virion is of serotype ShH10 and comprises a nucleic acid sequence encoding an RNA-guided endonuclease, and is for administration in conjunction with a second AAV vector virion of serotype ShH10, said second vector virion comprising a nucleic acid sequence encoding a guide RNA complementary to a target sequence from an aquaporin gene or a carbonic anhydrase gene and capable of directing said RNA-guided endonuclease to said target sequence.

The invention further provides an AAV vector virion for use in a method of modulating intracranial pressure or production of CSF, wherein said vector virion is of serotype ShH10 and comprises a nucleic acid sequence encoding a guide RNA complementary to a target sequence from an aquaporin gene or a carbonic anhydrase gene and is capable of directing an RNA-guided endonuclease to said target sequence; and is for administration in conjunction with a second AAV vector virion of serotype ShH10, said second vector virion comprising a nucleic acid sequence encoding said RNA-guided endonuclease.

The invention also provides the use of an AAV vector virion in the preparation of a medicament for modulating intraocular pressure or production of aqueous humour, wherein said vector virion is of serotype ShH10 and comprises a nucleic acid sequence encoding an RNA-guided endonuclease, and is for administration in conjunction with a second AAV vector virion of serotype ShH10, said second vector virion comprising a nucleic acid sequence encoding a guide RNA complementary to a target sequence from an aquaporin gene or a carbonic anhydrase gene and capable of directing said RNA-guided endonuclease to said target sequence.

The invention further provides the use of an AAV vector virion in the preparation of a medicament for modulating intraocular pressure or production of aqueous humour, wherein said vector virion is of serotype ShH10 and comprises a nucleic acid sequence encoding a guide RNA complementary to a target sequence from an aquaporin gene or a carbonic anhydrase gene and is capable of directing an RNA-guided endonuclease to said target sequence; and is for administration in conjunction with a second AAV vector virion of serotype ShH10, said second vector virion comprising a nucleic acid sequence encoding said RNA-guided endonuclease.

The invention also provides the use of an AAV vector virion in the preparation of a medicament for modulating intracranial pressure or production of CSF, wherein said vector virion is of serotype ShH10 and comprises a nucleic acid sequence encoding an RNA-guided endonuclease, and is for administration in conjunction with a second AAV vector virion of serotype ShH10, said second vector virion comprising a nucleic acid sequence encoding a guide RNA complementary to a target sequence from an aquaporin gene or a carbonic anhydrase gene and capable of directing said RNA-guided endonuclease to said target sequence.

The invention further provides the use of an AAV vector virion in the preparation of a medicament for modulating intracranial pressure or production of CSF, wherein said vector virion is of serotype ShH10 and comprises a nucleic acid sequence encoding a guide RNA complementary to a target sequence from an aquaporin gene or a carbonic anhydrase gene and is capable of directing an RNA-guided endonuclease to said target sequence; and is for administration in conjunction with a second AAV vector virion of serotype ShH10, said second vector virion comprising a nucleic acid sequence encoding said RNA-guided endonuclease.

The invention further provides a method of modulating intraocular pressure or production of aqueous humour, comprising administering first and second vector virions to a subject, wherein the first vector virion is of serotype ShH10 and comprises a nucleic acid sequence encoding a guide RNA complementary to a target sequence from an aquaporin gene or a carbonic anhydrase gene and is capable of directing an RNA-guided endonuclease to said target sequence; and the second AAV vector virion is of serotype ShH10 and comprises a nucleic acid sequence encoding said RNA-guided endonuclease.

The invention further provides a method of modulating intracranial pressure or production of CSF, comprising administering first and second vector virions to a subject, wherein the first vector virion is of serotype ShH10 and comprises a nucleic acid sequence encoding a guide RNA complementary to a target sequence from an aquaporin gene or a carbonic anhydrase gene and is capable of directing an RNA-guided endonuclease to said target sequence; and the second AAV vector virion is of serotype ShH10 and comprises a nucleic acid sequence encoding said RNA-guided endonuclease.

The virions may be administered in any suitable dose and the skilled person will be capable of determining an appropriate dose depending on the specific vector used and the clinical circumstances. For example, a single dose of 1×10⁷ to 1×10¹¹ genome copies (gc) of the or each vector may be suitable, e.g. 5×10⁷ to 5×10¹⁰ gc of the or each vector, e.g. 1×10⁸ to 1×10¹⁰ gc of the or each vector. However, lower titres may be possible.

When the vectors of the present invention are administered to the eye, such dosages have been found to provide good levels of ciliary body transduction with relatively low levels of transduction of Muller glial cells.

The terms “patient”, “subject” and “individual” may be used interchangeably. A subject to whom the compositions and method of the invention are applied will typically be a mammal, and may be a human or a non-human mammal, such as a non-human primate (e.g. ape, Old World monkey or New World monkey), livestock animal (e.g. bovine or porcine), companion animal (e.g. canine or feline) or laboratory animal such as a rodent (e.g. mouse or rat).

### DESCRIPTION OF THE FIGURES

FIG. 1. Analysis of published microarray data confirms Aquaporin 1, 4, 5 and Carbonic anhydrases 2, 3 and 14 are the most abundant isoforms in the mouse ciliary body. CEL data files were downloaded directly from the GEO repository (GSE10246—Lattin J E et al. Expression analysis of G Protein-Coupled Receptors in mouse macrophages. Immunome Res. 2008 Apr. 29; 4:5.) and imported into Partek Genomics Suite 6.6 (Partek Inc). The data was normalised using the Robust Multi-array Average background correction (Quantile normalisation summarising with median polish). A histogram of expression of all the AQP and CAR genes on the chip for Ciliary Body was then generated. Comparing across other ocular tissues AQP4, AQP5 and Car2, 3, 6, and 14 were particularly enriched in the ciliary body.

FIG. 2. Key aquaporin and carbonic anhydrase isoforms are detectable in the mouse ciliary body. Western blot analysis of A) dissected mouse ocular tissue identifies relative levels of Aqp1, 4 and Car2 protein between the cornea, retina and RPE. B) Quantitative PCR using Taqman probes on dissected mouse eye tissues shows comparable tissue levels of RNA transcripts. n=3 eyes per group.

FIG. 3. T7 endonuclease 1 genomic cleavage assay identifies several active SaCas9 sgRNA guides through in vitro testing. Genomic cleavage assay was performed three days after Lipofectamine 3000 transfection of different pAAV-SaCas9-sgRNA plasmids into a mouse B6-RPE cell line. The relative intensity of DNA cleavage products was assessed using a DNA1000 tapestation and allowed the calculation of relative genomic indel occurrence in the matching regions.

FIG. 4. Plasmid map and sequence of AAV-SaCas9 plasmid mAqp1 exon 1 guide 1B.

FIG. 5. Plasmid map and sequence of AAV-SaCas9 plasmid mAqp1 exon 1 guide 1E.

FIG. 6. Aquaporin 1 is expressed in the mouse ciliary body and can be disrupted by CRISPR-SaCas9. The mouse eye expresses aquaporin 1 (Aqp1) predominantly in the cornea, ciliary body and RPE. A) Representative Western blot with B) protein and C) quantitative PCR, n=3-4 eyes. D) Schematic of exon 1 of mouse Aqp1 displaying sequence and location of short guide RNAs (sgRNA) tested. E) T7 endonuclease 1 assay of plasmid transfected mouse Aqp1 sgRNA identifying the indel creation efficiency of each sgRNA labelled B and E were packaged into ShH10 serotype AAV vectors and used to infect the mouse B6-RPE cell line individually and in combination. F) T7 endonuclease 1 assay and G) quantitative PCR for mAqp1 was performed. Aqp1 expression was significantly reduced using sgRNA E alone and in combination with B (Mix). Kruskal-Wallis test with Dunn's multiple comparisons. ***p=0.001, ****p<0.001, n=10-12. H) Graphical representation of on-target genomic DNA alterations sequenced from Mix sgRNA treated B6-RPE cells demonstrating that complete excision of the intervening 83 bp region and a base insertion are the predominating changes using plasmid ligation and sequencing.

FIG. 7. CRISPR-Cas9 mediated disruption of ciliary body Aquaporin 1 lowers intraocular pressure in the mouse. Three weeks after intravitreal injection of 2×10¹⁰ genome copies of ShH10 virus encoding an equal proportion of mAqp1 B and E sgRNA (Mix) into one eye of a wildtype C57BL/6J mouse, A) T7 Endonuclease 1 assay demonstrates genomic DNA cleavage in ciliary body dissected from treated eyes alone. B) SaCas9 DNA is also detectable by PCR only in ciliary body tissue from injected eyes. C) Intraocular pressure (IOP) is reduced by mAqp1 disruption by a mean of 2.9 mmHg, paired t-test, n=18 pairs. D) IOP is not altered by control ShH10 CMV-GFP virus injection, One-way ANOVA, n=12-42 eyes. E) Representative Western blot and F) densitometry showing reduced Aqp1 protein in isolated ciliary body, paired t-test, p=0.009, n=7 pairs. No significant ‘off-target’ increase in thickness is seen in either the G) Cornea or H) Retina, paired t-test, n=9 pairs. UN=uninjected, MIX=ShH10-CMV-SaCas9-sgRNA B and E.

FIG. 8. Ciliary body aquaporin 1 disruption lowers intraocular pressure in two models of experimental glaucoma. Using the microbead model, data shown is pooled from three independent experiments of 3-5 mice per run. A) Intraocular pressure (IOP) of untreated (UN) and ShH10-CMV-SaCas9-sgRNA B and E (Mix) treated eyes. Injected one week after induction of ocular hypertension, treatment curtailed the increase in IOP. Two-way ANOVA, P=0.0003, n=12 mice. B) Analysis at the final timepoint showed a mean reduction in IOP of 3.9 mmHg, paired t-test, p=0.002, n=12 pairs. Dotted line represents mean baseline IOP at day zero of 12.7 mmHg. C) Representative Western blot of D) matched ex vivo ciliary body mAQP1 protein demonstrating reduced levels in treated eyes. Paired t-test, p=0.0008, n=12. Using a corticosteroid-induced ocular hypertension model, data shown is pooled from two independent experiments of 5 and 6 mice per run. E) IOP after 3 weeks of steroid induction is reduced in Mix treated eyes by a mean of 2.9 mmHg, paired t-test, p<0.0001, n=11. Dotted line represents mean baseline IOP at day zero of 11.3 mmHg. F) Representative Western blot of G) ex vivo ciliary body demonstrating reduced mAQP1 protein levels, paired t-test, p=0.0025, n=11.

FIG. 9. Human ciliary body expresses Aquaporin 1 and can be targeted by ShH10 virus to permit CRISPR-Cas9 mediated gene disruption. Human ex vivo ciliary body tissue surplus to corneal transplantation from six donors was obtained and could be maintained in culture for up to 7 days. A) Representative Western blots for aquaporin 1 (hAQP1) from two donors undergoing immediate dissection. B) Pooled qPCR expression data of tissue from all donors, n=2-6. AQP1 was enriched in ciliary body and corneal endothelium. Three Human sgRNA were generated similarly targeting exon 1 of hAQP1 and C) tested in 293T cells by plasmid transfection and T7 endonuclease 1 assay. D) sgRNA K was selected and packaged into ShH10 virus. Infecting 293T cells showed even higher indel formation rates by T7 endonuclease 1 assay. Human ciliary body was cultured with ShH10 virus expressing GFP under the control of the ubiquitous CMV promoter.

### DETAILED DESCRIPTION OF THE INVENTION

RNA-Guided Endonuclease and CRISPR System

The present invention uses the CRISPR (“clustered regularly interspaced short palindromic repeats”) system to modulate expression of target genes.

The CRISPR (or CRISPR-Cas) system is derived from a prokaryotic RNA-guided defence system. There are at least eleven different CRISPR-Cas systems, which have been grouped into three major types (I-III). Type II CRISPR-Cas systems have been adapted as a genome-engineering tool.

Most Type II CRISPR-Cas systems employ three components:
a protein endonuclease Cas (CRISPR-associated protein) having DNA nickase activity, which is referred to in this specification as an RNA-guided endonuclease (or an RNA-guided DNA endonuclease),
a “targeting” or “guide” RNA (CRISPR-RNA or crRNA) comprising a short sequence, typically of approximately 20 nucleotides, complementary to a target sequence (“protospacer”) in the target gene;
and a “scaffold” RNA (trans-acting CRISPR RNA or tracrRNA) which interacts with the crRNA and recruits the Cas endonuclease.

Assembly of these components and hybridisation of the crRNA with its target sequence in the chromosome results in cleavage of the chromosome by the endonuclease, typically at or close to the target sequence.

Cleavage also requires that the target DNA contains a recognition site for the Cas enzyme (protospacer adjacent motif, or PAM) located sufficiently close to the crRNA target sequence, typically immediately adjacent the 3′ end of the target sequence.

Cellular repair of the DNA break can lead to the insertion/deletion/mutation of bases and mutation at the target locus, often leading to inactivation of the locus.

This three-component system has been simplified by fusing together crRNA and tracrRNA, to create a chimeric single guide RNA (abbreviated as sgRNA or simply gRNA). Hybridisation of the sgRNA with the target sequence leads to cleavage of the target DNA at an adjacent/upstream PAM site. An sgRNA can therefore be regarded as comprising a crRNA component (which determines the target sequence) and a tracrRNA component (which recruits the endonuclease). Thus vectors for use as described here may encode multiple sgRNAs, each having the same tracrRNA component.

An example of a tracrRNA component from an sgRNA recognised by SaCas9 has the sequence:

GTTTTAGTACTCTGGAAACAGAATCTACTAAAACAAGGCAAAATGCCGTG
TTTATCTCGTCAACTTGTTGGCGAGATTTTT

and is used in the vectors described in the examples below.

The majority of type II CRISPR systems useful in the context of the present invention, the endonuclease is a Cas9 protein. Examples include Staphylococcus aureus (SaCas9), Streptococcus pyogenes (SpCas9), Neisseria meningitidis (NM Cas9), Streptococcus thermophilus (ST Cas9), Treponema denticola (TD Cas9), or variants thereof such as the D1135E, VRER, EQR or VQR variants of SpCas9.

PAM sequences recognised by these enzymes are as follows:

| Species/variant | PAM sequence |
|---|---|
| Streptococcus pyogenes (SpCas9) | NGG |
| SpCas9 D1135E variant | NGG |
| SpCas9 VRER variant | NGCG |
| SpCas9 EQR variant | NGAG |
| SpCas9 VQR variant | NGAN or NGNG |
| Streptococcus thermophilus | NNAGAAW |
| (ST Cas9) | |
| Treponema denticola (TD Cas9) | NAAAAC |
| Neisseria meningitidis (NM Cas9) | NNNNGATT |
| Staphylococcus aureus (SaCas9) | NNGRRT or |
| | NNGRR(N) |

Due to its relatively smaller size, SaCas9 and its variants may be preferred, in view of the limited coding capacity of an AAV genome. See, for example, Ran et al., Nature 520, 186-191 (2015) and references cited therein.

Certain CRISPR-Cas systems may not require tracrRNA for function. For example, Cpf1 is a single RNA-guided endonuclease of a class 2 CRISPR-Cas system which has been reported to mediate robust DNA interference with features distinct from Cas9, does not have a requirement for tracrRNA, and recognises a T-rich PAM sequence. It cleaves DNA via a staggered double-stranded break. See Zetsche et al., Cell, Volume 163(3), p 759-771, 22 October 2015, first published online 25 Sep. 2015.

In this specification, the term “guide RNA” will be used to encompass crRNA and sgRNA. Typically, the vectors of the invention will encode sgRNA directed to the relevant target site. However, vectors employing crRNA may also be used as long as a tracrRNA is also provided if the endonuclease requires it for function. Where required, the tracrRNA may be encoded by the same vector as the crRNA or the same vector as the endonuclease, as appropriate.

The protein component of the CRISPR system is referred to as an endonuclease and may have enzymatic activity (i.e. DNA nickase activity) when associated with the appropriate RNA factors. In such embodiments, the endonuclease will cleave chromosomal DNA at the relevant target site.

When using a catalytically active endonuclease, the target sequence recognised by the guide RNA may be in any portion of the gene where cleavage results in inactivation of the gene. In certain embodiments, it may be desirable that the target sequence is located in the transcribed portion of the gene, and optionally within the coding sequence.

If the case of aquaporin genes, especially AQP1, it may be desirable that the target sequence (or both target sequences, if two guide RNAs are employed) lies in the first exon sequence.

Certain approaches described here employ two different guide RNAs, to be delivered into a single cell, each directed to a different target sequence within the same gene. This may increase the efficiency of gene inactivation by causing deletion of a portion of the relevant gene. In such cases, both target sequences may be located in the transcribed portion of the same gene, and optionally both within the coding sequence of the same gene.

The cleavage sites specified by the guide RNAs may be separated by any suitable distance, e.g. greater than 1 kb, up to 1 kb, up to 500 bp or up to 250 bp, e.g. between 50 bp and 250 bp. They may be separated by at least 10 bp, at least 25 bp, at least 50 bp.

However, the endonuclease protein need not be enzymatically active. Catalytically inactive (or “dead”) endonuclease proteins may also be used in the context of the present invention, as they retain their ability to bind at the protospacer site targeted by the guide RNA. When bound, a catalytically dead endonuclease may sterically inhibit transcriptional initiation (e.g. when the protospacer site lies in the gene's promoter) or elongation (when it lies within an exon or intron). Alternatively, a catalytically dead endonuclease may be fused to a transcriptional repressor domain for inhibiting expression of the targeted gene. Such repressor domains may cause transcriptional repression or silencing via various mechanisms, including DNA methylation or heterochromatinisation, or histone deacetylation. Depending on the relevant mechanism, the endonuclease-repressor fusion may be targeted (by design of appropriate guide RNA) to different portions of the relevant gene, including the transcribed region (including exon and intron sequences), and regulatory sequences including the promoter and other transcription factor binding sites such as transcriptional enhancers.

Examples include the Kruppel associated box (KRAB) domain (e.g. from Kox1 protein), the CS (chromo shadow) domain of HP1α protein, the WRPW domain of Hes1 protein, MXI1 (Max-interacting protein), the mSin3 interacting domain, and the histone demethylase LSD1 (Lys-specific histone demethylase 1, which may be targeted to an enhancer region).

The endonuclease-repressor fusion may comprise a plurality of repressor domains. For example it may comprise multiple copies of the same repressor domain, e.g. 2, 3, 4 or 5 consecutive repeats of the same repressor domain. For example, a sequence of 4 concatenated mSin3 interacting domains is designated SID4X.

For more detail of transcriptional repressor domains and their use with catalytically dead endonucleases, see, for example, Gilbert et al., Cell 154, 442-451 (2013), Dominguez et al., Nature Reviews Molecular Cell Biology 17, 5-15, (2016), and references cited therein.

The term “endonuclease” is therefore used to encompass both catalytically active and catalytically dead proteins, unless the context demands otherwise. A catalytically dead endonuclease may be indicated by the prefix “d”, e.g. dCas, dCas9 or dCpf1. Use of a catalytically dead endonuclease to inhibit gene expression (whether or not in conjunction with a repressor domain) is often referred to as CRISPR interference or “CRISPRi”.

The endonuclease may comprise a nuclear localisation sequence (NLS) effective in mammalian cells, such as the SV40 Large T antigen NLS, which has the sequence PKKKRKV. Many other mammalian NLS sequences are known to the skilled person. The endonuclease may comprise multiple copies of an NLS, e.g. two or three copies of an NLS. Where multiple NLS sequences are present, they are typically repeats of the same NLS.

Typically, within an AAV vector genome, a gene encoding the endonuclease component of the system will be under the transcriptional control of an RNA polymerase II promoter, e.g. a viral or human RNA polymerase II promoter. Examples include the cytomegalovirus (CMV) or SV40 promoter, or a mammalian “housekeeping” promoter. Genes encoding any RNA components (sgRNA, crRNA or tracrRNA) will typically be under the transcriptional control of an RNA polymerase III promoter (e.g. a human RNA polymerase UII promoter) such as the U6 or H1 promoter, or variants thereof which retain or have enhanced activity.

In some circumstances, it can be beneficial to employ multiple vector virions carrying different payloads, largely because of the constraints imposed by the relatively restricted size of genome which an AAV vector can carry, discussed above.

For example, delivering two different guide RNAs into a single cell, each directed to a different target sequence within the same gene, may increase the efficiency of gene inactivation by causing deletion of a portion of the relevant gene. However, because of size constraints, it may not be possible for a single vector genome to encode an endonuclease and multiple guide RNAs. One possible solution involves employing two vector virions, each encoding an endonuclease and one guide RNA. Another involves employing one virion encoding the endonuclease and another encoding two (or more) guide RNAs. The first option (two vector virions each encoding an endonuclease and one guide RNA) may be more attractive, as each virion alone carries the full CRISPR apparatus and so should be capable of downregulating expression of the target gene. The “split” approach of separating endonuclease and guide RNAs into different vectors relies on cell being transduced by one vector of each type to achieve downregulation. Transduction by just one virion alone will have no effect.

Alternatively, use of a large endonuclease may require the endonuclease to be encoded by one vector and the guide RNA (or guide RNAs) to be encoded by another. For example, if a CRIPSRi approach is employed, in which the endonuclease comprises a transcriptional repressor domain, the AAV vector genome may not have sufficient capacity also to encode a guide RNA, necessitating use of a further vector encoding the guide RNA.

Adeno-Associated Virus (AAV) Vectors

Adeno-associated virus (AAV) is a replication-deficient parvovirus, the single stranded DNA genome of which is about 4.7 kb in length including 145 nucleotide inverted terminal repeat (ITRs). The nucleotide sequence of the AAV serotype 2 (AAV2) genome is presented in Srivastava el al., J Virol, 45: 555-564 (1983) as corrected by Ruffing el al., J Gen Virol, 75: 3385-3392 (1994). Cis-acting sequences directing viral DNA replication (rep), encapsidation/packaging and host cell chromosome integration are contained within the ITRs. Three AAV promoters (named p5, pI9, and p40 for their relative map locations) drive the expression of the two AAV internal open reading frames encoding rep and cap genes. The two rep promoters (p5 and p i9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. VP2 and VP3 are progressively shorter versions of the VP1 protein, having the same C-terminus but lacking progressively longer amounts of sequence from the N-terminus of VP1.

As the signals directing AAV replication, genome encapsidation and integration are contained within the ITRs of the AAV genome, some or all of the internal sequence of the genome (encoding replication and structural capsid proteins, rep-cap) may be replaced with foreign DNA such as an expression cassette, with the rep and cap proteins provided in trans. The sequence located between ITRs of an AAV vector genome is referred to herein as the “payload”.

The actual capacity of any particular AAV particle may vary depending on the viral proteins employed. Typically, the vector genome (including ITRs) is between about 0.7 kb and about 5 kb, e.g. not more than about 5 kb, e.g. not more than about 4.9 kb, 4.8 kb or 4.7 kb.

Wild type AAV ITRs are each typically 145 bases in length, although shorter sequences may also be functional. For example, the vectors described in the examples below utilise sequences of 130 bases which are functionally equivalent to wild type AAV2 ITRs. Thus, the payload is typically not more than about 4.7 kb, 4.6 kb, 4.5 kb or 4.4 kb in length. Preferably it is not more than 4.4. kb in length.

A recombinant AAV (rAAV) may therefore contain up to about 4.7 kb, 4.6 kb, 4.5 kb or 4.4 kb of unique payload sequence.

Following infection of a target cell, protein expression and replication from the vector requires synthesis of a complementary DNA strand to form a double stranded genome. This second strand synthesis represents a rate limiting step in transgene expression.

The requirement for second strand synthesis can be avoided using so-called “self complementary AAV” (scAAV) vectors in which the payload contains two copies of the same transgene payload in opposite orientations to one another, i.e. a first payload sequence followed by the reverse complement of that sequence. These scAAV genomes are capable of adopting either a hairpin structure, in which the complementary payload sequences hybridise intramolecularly with each other, or a double stranded complex of two genome molecules hybridised to one another. Transgene expression from such scAAVs is much more efficient than from conventional rAAVs, but the effective payload capacity of the vector genome is halved because of the need for the genome to carry two complementary copies of the payload sequence.

The genes encoding RNA-guided endonucleases are typically too large to be housed in scAAV vectors, although scAAV vectors may find use to carry guide RNA sequences (and tracrRNAs if required), with the endonuclease provided in trans from a separate vector.

An scAAV vector genome may contain one or more mutations in one of the ITR sequences to inhibit resolution at one terminal repeat, and consequently increase yield in an scAAV preparation. Thus one of the ITRs in an scAAV may be deleted for the terminal resolution site or may contain an inactivating mutation in the terminal resolution site. See, for example, Wang et al., Gene Therapy (2003) 10, 2105-2111 and McCarty et al., Gene Therapy (2003) 10, 2112-2118. It will therefore be apparent that the two ITR sequences at either end of an AAV genome need not be identical.

scAAVs are reviewed in McCarty, Molecular Therapy, 16(10), 2008, 1648-1656.

In this specification, the term “rAAV vector” is generally used to refer to vectors having only one copy of any given payload sequence (i.e. a rAAV vector is not an scAAV vector), and the term “AAV vector” is used to encompass both rAAV and scAAV vectors.

AAV sequences in the AAV vector genomes (e.g. ITRs) may be from any AAV serotype for which a recombinant virus can be derived including, but not limited to, AAV serotypes AAV1, AAV2, AAV3, AAV4, AAV5, AAV6, AAV7, AAV8, AAV9, AAV10, AAV11 and AAV PHP.B. The nucleotide sequences of the genomes of the AAV serotypes are known in the art. For example, the complete genome of AAV1 is provided in GenBank Accession No. NC_002077; the complete genome of AAV2 is provided in GenBank Accession No. NC 001401 and Srivastava et al., J. Virol, 45: 555-564 {1983); the complete genome of AAV3 is provided in GenBank Accession No. NC_1829; the complete genome of AAV-4 is provided in GenBank Accession No. NC_001829; the AAV5 genome is provided in GenBank Accession No. AF085716; the complete genome of AAV6 is provided in GenBank Accession No. NC_00 1862; at least portions of AAV7 and AAV8 genomes are provided in GenBank Accession Nos. AX753246 and AX753249, respectively; the AAV9 genome is provided in Gao et al., J. Virol., 78: 6381-6388 (2004); the AAV-10 genome is provided in Mol. Ther., 13(1): 67-76 (2006); the AAV11 genome is provided in Virology, 330(2): 375-383 (2004); AAV PHP.B is described by Deverman et al., Nature Biotech. 34(2), 204-209 and its sequence deposited under GenBank Accession No. KU056473.1.

The ITR sequences may be from any suitable AAV type. For example, they may be from AAV2, or be functional equivalents thereof. The scAAV vectors described in the examples below contain ITRs which are functionally equivalent to wild type AAV2 ITRs and have the sequences:

CCTGCAGGCAGCTGCGCGCTCGCTCGCTCACTGAGGCCGCCCGGGCGTCG
GGCGACCTTTGGTCGCCCGGCCTCAGTGAGCGAGCGAGCGCGCAGAGAGG
GAGTGGCCAACTCCATCACTAGGGGTTCCT
and
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAGCTGCCTGCAGG

An AAV vector may have genomic ITRs from a first serotype (“A”) and proteins from a second serotype (“B”). Such a vector may be referred to as type “AAV A/B”. However, since the viral proteins largely determine the serological properties of the virion particle, such a vector may still be referred to as being of serotype B. Thus, the vectors described in the example are of type 2/ShH10, but are generally regarded as being of serotype ShH10 because of their capsid proteins.

Virion particles comprising vector genomes of the invention are typically generated in packaging cells capable of replicating viral genomes, expressing viral proteins (e.g. rep and cap proteins), and assembling virion particles. Packaging cells may also require helper virus functions, e.g. from adenovirus, El-deleted adenovirus or herpesvirus. Techniques to produce AAV vector particles in packaging cells are standard in the art. Production of pseudotyped AAV is disclosed in, for example, WO 01/83692. In various embodiments, AAV capsid proteins may be modified to enhance delivery of the recombinant vector. Modifications to capsid proteins are generally known in the art. See, for example, US 2005/0053922 and US 2009/0202490.

One method of generating a packaging cell is to create a cell line that stably expresses all the necessary components for AAV particle production. For example, a plasmid (or multiple plasmids) comprising an AAV genome lacking AAV rep and cap genes, AAV rep and cap genes separate from the AAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of AAV. Other examples of suitable methods employ adenovirus or baculovirus rather than plasmids to introduce AAV genomes and/or rep and cap genes into packaging cells.

Alternatively, a packaging cell can be generated by simply transforming a suitable cell with one or more plasmids encoding an AAV genome, AAV proteins, and any required helper virus functions. The so-called “triple transfection” method utilises three plasmids each carrying one of these sets of genes. See Grieger et al., Nature Protocols 1(3), 1412-128 (2006) and references cited therein.

General principles of AAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

Techniques for scAAV production are described by Grieger et al., Molecular Therapy 24(2), 287-297, 2016.

The invention thus provides a packaging cell capable of producing any of the individual infectious AAV virion particles described herein. The packaging cell is typically a eukaryotic cell, such as a mammalian cell, e.g. a primate cell, e.g. a human cell. Typically it is a cell line. In one embodiment packaging cells may be stably transformed cancer cells such as HeLa cells, 293 cells (HEK293 or HEK293T cells) and PerC.6 cells (a cognate 293 line). In another embodiment, packaging cells are cells that are not transformed cancer cells such as low passage 293 cells (human fetal kidney cells transformed with El of adenovirus), MRC-5 cells (human fetal fibroblasts), WI-38 cells (human fetal fibroblasts), Vero cells (monkey kidney cells) and FRhL-2 cells (rhesus fetal lung cells).

ShH10 Serotype

The AAV virion particles of the invention are of the ShH10 serotype, as described by Klimcsak et al., (2009) A Novel Adeno-Associated Viral Variant for Efficient and Selective Intravitreal Transduction of Rat Muller Cells. PLoS ONE 4(10): e7467 (doi:10.1371/journal.pone.0007467). This serotype has been found to provide efficient and specific transduction of the ciliary body and choroid plexus.

The main determinants of serotype in AAV vector virions are the capsid proteins. The published sequence for the ShH10 capsid protein VP1 is follows, and will be referred to here as the “native” ShH10 VP1 sequence:

```
VP1:
    1 MAADGYLPDW LEDNLSEGIR EWWDLKPGAP KPKANQQKQD
        DGRGLVLPGY KYLGPFNGLD
  61 KGEPVNAADA AALEHDKAYD QQLKAGDNPY LRYNHADAEF
        QERLQEDTSF GGNLGRAVFQ
121 AKKRVLEPFG LVEEGAKTAP GKKRPVEQSP QEPDSSSGIG
        KTGQQPAKKR LNFGQTGDSE
181 SVPDPQPLGE PPATPAAVGP TTMASGGGAP MADNNEGADG
        VGNASGNWHC DSTWLGDRVI
241 TTSTRTWALP TYNNHLYKQI SSASTGASND NHYFGYSTPW
        GYFDFNRFHC HFSPRDWQRL
301 INNNWGFRPK RLNFKLFNVQ VKEVTTNDGV TTIANNLTST
        VQVFSDSEYQ LPYVLGSAHQ
361 GCLPPFPADV FMIPQYGYLT LNNGSQAVGR SSFYCLEYFP
        SQMLRTGNNF TFSYTFEDVP
421 FHSSYAHSQS LDRLMNPLID QYLYYLNRTQ DQSGSAQNKD
        LLFSRGSPAG MSVQPKNWLP
481 GPCYRQQRVS KTKTDNNNSN FTWTGASKYN LNGRESIINP
        GTAMASHKDD KNKFFPMSGV
541 MIFGKESAGA SNTALDNVMI TDEEEIKATN PVATERFGTV
        AVNLQSSSTD PATGDVHVMG
601 ALPGMVWQDR DVYLQGPIWA KIPHTDGHFH PSPLMGGFGL
        KNPPPQILIK NTPVPANPPA
661 EFSATKFASF ITQYSTGQVS VEIEWELQKE NSKRWNPEVQ
        YTSNYAKSAN VDFTVDNNGL
721 YTEPRPIGTR YLTRPL
```

This sequence is identical to the VP1 capsid protein of AAV6 except for the residues V319 (I in AAV6), D451 (N in AAV6), N532 (D in AAV6) and N642 (H in AAV6).

By analogy with AAV6 (Rutledge et al., J. Virol., 72(1), 309-319, 1998), the native sequences of VP2 and VP3 are believed to be as follows:

```
VP2:
    1 TAPGKKRPVE QSPQEPDSSS GIGKTGQQPA KKRLNFGQTG
        DSESVPDPQP LGEPPATPAA
  61 VGPTTMASGG GAPMADNNEG ADGVGNASGN WHCDSTWLGD
        RVITTSTRTW ALPTYNNHLY
121 KQISSASTGA SNDNHYFGYS TPWGYFDFNR FHCHFSPRDW
        QRLINNNWGF RPKRLNFKLF
181 NVQVKEVTTN DGVTTIANNL TSTVQVFSDS EYQLPYVLGS
        AHQGCLPPFP ADVFMIPQYG
241 YLTLNNGSQA VGRSSFYCLE YFPSQMLRTG NNFTFSYTFE
        DVPFHSSYAH SQSLDRLMNP
301 LIDQYLYYLN RTQDQSGSAQ NKDLLFSRGS PAGMSVQPKN
        WLPGPCYRQQ RVSKTKTDNN
361 NSNFTWTGAS KYNLNGRESI INPGTAMASH KDDKNKFFPM
        SGVMIFGKES AGASNTALDN
421 VMITDEEEIK ATNPVATERF GTVAVNLQSS STDPATGDVH
        VMGALPGMVW QDRDVYLQGP
481 IWAKIPHTDG HFHPSPLMGG FGLKNPPPQI LIKNTPVPAN
        PPAEFSATKF ASFITQYSTG
541 QVSVEIEWEL QKENSKRWNP EVQYTSNYAK SANVDFTVDN
        NGLYTEPRPI GTRYLTRPL
VP3:
    1 MASGGGAPMA DNNEGADGVG NASGNWHCDS TWLGDRVITT
        STRTWALPTY NNHLYKQISS
  61 ASTGASNDNH YFGYSTPWGY FDFNRFHCHF SPRDWQRLIN
        NNWGFRPKRL NFKLFNVQVK
121 EVTTNDGVTT IANNLTSTVQ VFSDSEYQLP YVLGSAHQGC
        LPPFPADVFM IPQYGYLTLN
181 NGSQAVGRSS FYCLEYFPSQ MLRTGNNFTF SYTFEDVPFH
        SSYAHSQSLD RLMNPLIDQY
241 LYYLNRTQDQ SGSAQNKDLL FSRGSPAGMS VQPKNWLPGP
        CYRQQRVSKT KTDNNNSNFT
301 WTGASKYNLN GRESIINPGT AMASHKDDKN KFFPMSGVMI
        FGKESAGASN TALDNVMITD
361 EEEIKATNPV ATERFGTVAV NLQSSSTDPA TGDVHVMGAL
        PGMVWQDRDV YLQGPIWAKI
421 PHTDGHFHPS PLMGGFGLKN PPPQILIKNT PVPANPPAEF
        SATKFASFIT QYSTGQVSVE
481 IEWELQKENS KRWNPEVQYT SNYAKSANVD FTVDNNGLYT
        EPRPIGTRYL TRPL
```

Thus the AAV virions of the invention typically comprise a VP1 capsid protein having the native VP1 sequence shown above or having at least 90% identity thereto. The VP1 capsid protein may have at least 90%, 96%, 97%, 98% or 99% identity to the native sequence. It will typically be desirable that the VP2 capsid protein contains one, two, three or all four of the residues V319, D451, N532 and N642, and preferably all four of these residues.

Additionally or alternatively, the AAV virions of the invention typically comprise a VP2 capsid protein having the native VP2 sequence shown above or having at least 90% identity thereto. The VP2 capsid protein may have at least 90%, 96%, 97%, 98% or 99% identity to the native sequence. It will typically be desirable that the VP2 capsid protein contains one, two, three or all four of the residues V182, D314, N395 and N505, and preferably all four of these residues.

Additionally or alternatively, the AAV virions of the invention typically comprise a VP3 capsid protein having the native VP3 sequence shown above or having at least 90% identity thereto. The VP3 capsid protein may have at least 90%, 96%, 97%, 98% or 99% identity to the native sequence. It will typically be desirable that the VP3 capsid protein contains one, two, three or all four of the residues V117, D249, N330 and N440, and preferably all four of these residues.

Typically, all three of the VP1, VP2 and VP3 proteins have at least 90% identity to the respective native sequences, e.g. at least 90%, 96%, 97%, 98% or 99% identity to the native sequence.

Additionally or alternatively, all three of the VP1, VP2 and VP3 proteins may contain one, two, three or all four of the residues V117, D249, N330 and N440 (as numbered in the VP1 sequence), and preferably all four of these residues.

Percent (%) amino acid sequence identity between a candidate sequence and the reference sequences presented above is defined as the percentage of amino acid residues in the candidate sequence that are identical with the amino acid residues in the reference sequence, after aligning the sequences and introducing gaps, if necessary, to achieve the optimum alignment, and not considering any conservative substitutions as part of the sequence identity. % identity values may be determined by WU-BLAST-2 (Altschul et al., Methods in Enzymology, 266:460-480 (1996)). WU-BLAST-2 uses several search parameters, most of which are set to the default values. The adjustable parameters are set with the following values: overlap span=1, overlap fraction=0.125, word threshold (T)=11. A % amino acid sequence identity value is determined by the number of matching identical residues as determined by WU-BLAST-2, divided by the total number of residues of the reference sequence (gaps introduced by WU-BLAST-2 into the reference sequence to maximize the alignment score being ignored), multiplied by 100.

A conservative substitution may be defined as a substitution within an amino acid class and/or a substitution that scores positive in the BLOSUM62 matrix.

According to one classification, the amino acid classes are acidic, basic, uncharged polar and nonpolar, wherein acidic amino acids are Asp and Glu; basic amino acids are Arg, Lys and His; uncharged polar amino acids are Asn, Gln, Ser, Thr and Tyr; and non-polar amino acids are Ala, Gly, Val, Leu, Ile, Pro, Phe, Met, Trp and Cys.

According to another classification, the amino acid classes are small hydrophilic, acid/acid amide/hydrophilic, basic, small hydrophobic and aromatic, wherein small hydrophilic amino acids are Ser, Thr, Pro, Ala and Gly; acid/acidamide/hydrophilic amino acids are Asn, Asp, Glu and Gln; basic amino acids are His, Arg and Lys; small hydrophobic amino acids are Met, Ile, Leu and Val; and aromatic amino acids are Phe, Tyr and Trp

Substitutions which score positive in the BLOSUM62 matrix are as follows:

| | | | | | | | | | | | | | | | | | | | | |
|---|---|---|---|---|---|---|---|---|---|---|---|---|---|---|---|---|---|---|---|---|
| Original | C | S | T | P | A | G | N | D | E | Q | H | R | K | M | I | L | V | F | Y | W |
| Residue | | | | | | | | | | | | | | | | | | | | |
| Substitution | — | T | S | — | S | — | S | N | D | E | N | Q | E | I | M | M | M | Y | H | F |
| | | A | | | | | D | E | Q | R | Y | K | Q | L | L | I | I | W | F | Y |
| | | N | | | | | H | | K | K | | | R | V | V | V | L | | W | |

Aquaporins (AQP)

Aquaporins are integral membrane proteins which facilitate transport of water molecules across biological membranes. They share a common overall structure, with a bundle of six transmembrane helices connected by 5 loop regions, two of which possess a conserved asparagine-proline-alanine (NPA) motif, one located on each side of the membrane.

Due to their role in water transport, they are implicated in various functions involving production of extracellular fluids such as aqueous humour and CSF. AQP1 knock-out mice show reduced IOP compared to normal mice (Zhang et al., J. Gen. Physiol., 2002, 119: 561-569) and siRNA against AQP4 has been proposed as a therapy for lowering IOP (WO2008/067382) but without any demonstration of efficacy.

Mammals are believed to possess 13 different aquaporin genes. Details of the human and murine aquaporin genes are shown in Tables 1 and 2 below.

Aquaporins AQP1, AQP2, AQP3, AQP4, AQP5, AQP6, AQP7 and AQP11, at least, are believed to be expressed in the ciliary body and/or choroid plexus. Any of these may therefore represent targets for treatment.

In the ciliary body, AQP1, AQP4 and AQP5 are the most highly expressed aquaporins and may represent particularly good targets for ocular conditions.

AQP1 and AQP4 are also highly expressed in the choroid plexus, so may be particularly good targets for cerebral conditions.

TABLE 1
Homo sapiens AQP genes

| | | | Nucleotide | | |
|---|---|---|---|---|---|
| GeneID | Symbol | Description | Accession no | Map_Location | Chromosome |
| 358 | AQP1 | aquaporin 1 | NC_000007.14 | 7p14.3 | 7 |
| | | (Colton blood | | | |
| | | group) | | | |
| 359 | AQP2 | aquaporin 2 | NC_000012.12 | 12q13.12 | 12 |
| 360 | AQP3 | aquaporin 3 | NC_000009.12 | 9p13.3 | 9 |
| | | (Gill blood | | | |
| | | group) | | | |
| 361 | AQP4 | aquaporin 4 | NC_000018.10 | 18q11.2 | 18 |
| 362 | AQP5 | aquaporin 5 | NC_000012.12 | 12q13.12 | 12 |
| 363 | AQP6 | aquaporin 6 | NC_000012.12 | 12q13.12 | 12 |
| 364 | AQP7 | aquaporin 7 | NC_000009.12 | 9p13.3 | 9 |
| 343 | AQP8 | aquaporin 8 | NC_000016.10 | 16p12.1 | 16 |
| 366 | AQP9 | aquaporin 9 | NC_000015.10 | 15q21.3 | 15 |
| 89872 | AQP10 | aquaporin 10 | NC_000001.11 | 1q21.3 | 1 |
| 282679 | AQP11 | aquaporin 11 | NC_000011.10 | 11q14.1 | 11 |
| 375318 | AQP12A | aquaporin 12A | NC_000002.12 | 2q37.3 | 2 |
| 653437 | AQP12B | aquaporin 12B | NC_000002.12 | 2q37.3 | 2 |

| | Start Position on | End Position on | | |
|---|---|---|---|---|
| Symbol | Genomic Accession | Genomic Accession | Orientation | Exon Count |
| AQP1 | 30911800 | 30925516 | plus | 7 |
| AQP2 | 49950741 | 49958881 | plus | 4 |
| AQP3 | 33441154 | 33447633 | minus | 6 |
| AQP4 | 26852038 | 26865844 | minus | 6 |
| AQP5 | 49961496 | 49965682 | plus | 5 |
| AQP6 | 49972837 | 49977139 | plus | 4 |
| AQP7 | 33383135 | 33402682 | minus | 10 |
| AQP7P1 | 63315243 | 63334520 | minus | 4 |
| AQP7P2 | 64621504 | 64639027 | minus | 0 |
| AQP7P3 | 65877574 | 65912560 | plus | 4 |
| AQP7P4 | 63133597 | 63143456 | plus | 0 |
| AQP7P5 | 40463754 | 40499011 | minus | 16 |
| AQP8 | 25216917 | 25228932 | plus | 6 |
| AQP9 | 58138169 | 58185911 | plus | 6 |
| AQP10 | 1.54E+08 | 1.54E+08 | plus | 6 |
| AQP11 | 77589635 | 77610356 | plus | 3 |
| AQP12A | 2.41E+08 | 2.41E+08 | plus | 4 |
| AQP12B | 2.41E+08 | 2.41E+08 | minus | 7 |

TABLE 2
Mus musculus AQP genes

| | | | Nucleotide | | |
|---|---|---|---|---|---|
| GeneID | Symbol | Description | Accession No | Map_Location | Chromosome |
| 11826 | Aqp1 | aquaporin 1 | NC_000072.6 | 6 27.38 cM | 6 |
| 11827 | Aqp2 | aquaporin 2 | NC_000081.6 | 15 56.13 cM | 15 |
| 11828 | Aqp3 | aquaporin 3 | NC_000070.6 | 4 A5 | 4 |
| 11829 | Aqp4 | aquaporin 4 | NC_000084.6 | 18 8.74 cM | 18 |
| 11830 | Aqp5 | aquaporin 5 | NC_000081.6 | 15 56.13 cM | 15 |
| 11831 | Aqp6 | aquaporin 6 | NC_000081.6 | 15 56.13 cM | 15 |
| 11832 | Aqp7 | aquaporin 7 | NC_000070.6 | 4 A5 | 4 |
| 11833 | Aqp8 | aquaporin 8 | NC_000073.6 | 7 F3 | 7 |
| 64008 | Aqp9 | aquaporin 9 | NC_000075.6 | 9 D | 9 |
| 435743 | Aqp10-s | aquaporin 10, | NC_000069.6 | 3 F1 | 3 |
| | | pseudogene | | | |
| 66333 | Aqp11 | aquaporin 11 | NC_000073.6 | 7 E1 | 7 |
| 208760 | Aqp12 | aquaporin 12 | NC_000067.6 | 1 D | 1 |

| | Start Position on | End Position on | | |
|---|---|---|---|---|
| Symbol | Genomic Accession | Genomic Accession | Orientation | Exon Count |
| Aqp1 | 55336299 | 55348555 | plus | 4 |
| Aqp2 | 99579056 | 99584545 | plus | 4 |
| Aqp3 | 41092724 | 41098183 | minus | 6 |
| Aqp4 | 15389394 | 15410982 | minus | 10 |
| Aqp5 | 99591028 | 99594829 | plus | 4 |
| Aqp6 | 99600689 | 99605477 | plus | 5 |
| Aqp7 | 41033074 | 41048295 | minus | 11 |
| Aqp8 | 1.23E+08 | 1.23E+08 | plus | 6 |
| Aqp9 | 71110659 | 71168085 | minus | 11 |
| Aqp10-ps | 89963444 | 89969734 | minus | 0 |
| Aqp11 | 97726379 | 97738247 | minus | 3 |
| Aqp12 | 93006334 | 93012269 | plus | 3 |

Carbonic Anhydrases (CAR)

Carbonic anhydrases are a family of enzymes which catalyse the interconversion of carbon dioxide and water to bicarbonate and protons. Topical carbonic anhydrase inhibitors such as acetazolamide, methazolamide, dorzolamide and brinzolamide are used in the treatment of glaucoma, primarily because of their inhibitory effect on the production of aqueous humour.

The cellular locations of the enzymes are variable. They may be cytosolic (CARs 1, 2, 3, 7 and 13), mitochondrial (CARs 5a and 5b), secreted (CAR6), or membrane associated (CARs 4, 9, 12 and 14, and 15 in species other than humans and chimpanzees). The functions of CARs 8, 10 and 11 remain unclear and they may not have catalytic activity. CAR15 appears not to be expressed in humans and chimpanzees.

Details of the human and murine carbonic anhydrase genes are shown in Tables 3 and 4 below. CAR2, CAR3, CAR4, CR5b, CAR6, CARE, CAR9, CAR10, CAR12 and CAR14 are believed to be expressed in the ciliary body and/or choroid plexus. CAR15 is also expressed in the ciliary body in mouse. CAR2, CAR3 and CAR14 are believed to be the most highly expressed in the ciliary body and so represent good targets. A previous study involving the use of siRNA for reducing carbonic anhydrase in glaucoma (Jimenez et al., RNAi: A New Strategy for Treating Ocular Hypertension Silencing Carbonic Anhydrases. ARVO Abnr 405, 2006) reported that siRNAs against CAR2, CAR4 and CAR12 lowered IOP in rabbits. Thus CAR4 and CAR12 may also represent good targets.

CAR2, CAR4 and CAR12 are believed to be the most highly expressed in choroid plexus.

TABLE 3
Homo sapiens CAR genes

| | | | Nucleotide | Map | |
|---|---|---|---|---|---|
| GeneID | Symbol | Description | Accession No | Location | Chromosome |
| 759 | CA1 | carbonic anhydrase 1 | NC_000008.11 | 8q21.2 | 8 |
| 760 | CA2 | carbonic anhydrase 2 | NC_000008.11 | 8q21.2 | 8 |
| 761 | CA3 | carbonic anhydrase 3 | NC_000008.11 | 8q21.2 | 8 |
| 762 | CA4 | carbonic anhydrase 4 | NC_000017.11 | 17q23.1 | 17 |
| 763 | CA5A | carbonic anhydrase 5A | NC_000016.10 | 16q24.2 | 16 |
| 764 | CA5AP1 | carbonic anhydrase 5A | NC_000016.10 | 16p11.2 | 16 |
| | | pseudogene 1 | | | |
| 11238 | CA5B | carbonic anhydrase 5B | NC_000023.11 | Xp22.2 | X |
| | | carbonic anhydrase 5B | | | |
| 340591 | CA5BP1 | pseudogene 1 | NC_000023.11 | Xp22.2 | X |
| 765 | CA6 | carbonic anhydrase 6 | NC_000001.11 | 1p36.23 | 1 |
| 766 | CA7 | carbonic anhydrase 7 | NC_000016.10 | 16q22.1 | 16 |
| 767 | CA8 | carbonic anhydrase 8 | NC_000008.11 | 8q12.1 | 8 |
| 768 | CA9 | carbonic anhydrase 9 | NC_000009.12 | 9p13.3 | 9 |
| 56934 | CA10 | carbonic anhydrase 10 | NC_000017.11 | 17q21.33-q22 | 17 |
| 770 | CA11 | carbonic anhydrase 11 | NC_000019.10 | 19q13.33 | 19 |
| 771 | CA12 | carbonic anhydrase 12 | NC_000015.10 | 15q22.2 | 15 |
| 377677 | CA13 | carbonic anhydrase 13 | NC_000008.11 | 8q21.2 | 8 |
| 23632 | CA14 | carbonic anhydrase 14 | NC_000001.11 | 1q21.2 | 1 |
| 100996435 | CA15P1 | carbonic anhydrase 15 | | | 22 |
| | | pseudogene 1 | | | |
| 440795 | CA15P2 | carbonic anhydrase 15 | NC_000022.11 | 22q11.21 | 22 |
| | | pseudogene 2 | | | |
| 100996556 | CA15P3 | carbonic anhydrase 15 | | 22q11.21 | 22 |
| | | pseudogene 3 | | | |

| | Start Position on | End Position on | | |
|---|---|---|---|---|
| Symbol | Genomic Accession | Genomic Accession | Orientation | Exon Count |
| CA1 | 85328229 | 85378154 | minus | 11 |
| CA2 | 85463902 | 85481492 | plus | 7 |
| CA3 | 85438827 | 85449040 | plus | 7 |
| CA4 | 60149941 | 60178118 | plus | 12 |
| CA5A | 87888019 | 87936575 | minus | 10 |
| CA5AP1 | 29618675 | 29636331 | minus | 0 |
| CA5B | 15738281 | 15787625 | plus | 11 |
| CA5BP1 | 15674916 | 15703351 | plus | 5 |
| CA6 | 8945786 | 8975092 | plus | 10 |
| CA7 | 66844379 | 66854149 | plus | 9 |
| CA8 | 60185420 | 60281423 | minus | 14 |
| CA9 | 35673859 | 35681159 | plus | 11 |
| CA10 | 51630313 | 52160017 | minus | 12 |
| CA11 | 48637942 | 48646312 | minus | 9 |
| CA12 | 63321378 | 63382166 | minus | 11 |
| CA13 | 85245487 | 85284073 | plus | 7 |
| CA14 | 1.5E+08 | 1.5E+08 | plus | 11 |
| CA15P1 | | | | |
| CA15P2 | 18635987 | 18637497 | minus | 0 |
| CA15P3 | | | | |

TABLE 4
Mus musculus CAR genes

| | | | Nucleotide | Map | |
|---|---|---|---|---|---|
| GeneID | Symbol | Description | Accession No | Location | Chromosome |
| 12346 | Car1 | carbonic anhydrase 1 | NC_000069.6 | 3 3.18 cM | 3 |
| 12349 | Car2 | carbonic anhydrase 2 | NC_000069.6 | 3 3.23 cM | 3 |
| 12350 | Car3 | carbonic anhydrase 3 | NC_000069.6 | 3 3.22 cM | 3 |
| 12351 | Car4 | carbonic anhydrase 4 | NC_000077.6 | 11 C | 11 |
| 12352 | Car5a | carbonic anhydrase | NC_000074.6 | 8 70.81 cM | 8 |
| | | 5a, mitochondrial | | | |
| 56078 | Car5b | carbonic anhydrase | NC_000086.7 | X F5 | X |
| | | 5b, mitochondria | | | |
| 12353 | Car6 | carbonic anhydrase 6 | NC_000070.6 | 4 E2 | 4 |
| 12354 | Car7 | carbonic anhydrase 7 | NC_000074.6 | 8 D3 | 8 |
| 12319 | Car8 | carbonic anhydrase 8 | NC_000070.6 | 4 3.53 cM | 4 |
| 230099 | Car9 | carbonic anhydrase 9 | NC_000070.6 | 4 A5 | 4 |
| 72605 | Car10 | carbonic anhydrase 10 | NC_000077.6 | 11 D | 11 |
| 12348 | Car11 | carbonic anhydrase 11 | NC_000073.6 | 7 B3 | 7 |
| 76459 | Car12 | carbonic anhydrase 12 | NC_000075.6 | 9 C | 9 |
| 71934 | Car13 | carbonic anhydrase 13 | NC_000069.6 | 3 A1 | 3 |
| 23831 | Car14 | carbonic anhydrase 14 | NC_000069.6 | 3 F2.1 | 3 |
| 80733 | Car15 | carbonic anhydrase 15 | NC_000082.6 | 16 11.05 cM | 16 |

| | Start Position on | End Position on | | |
|---|---|---|---|---|
| Symbol | Genomic Accession | Genomic Accession | Orientation | Exon Count |
| Car1 | 14766214 | 14608365 | minus | 9 |
| Car2 | 14886278 | 14900770 | plus | 8 |
| Car3 | 14863538 | 14872381 | plus | 7 |
| Car4 | 84957754 | 84966054 | plus | 9 |
| Car5a | 1.22E+08 | 1.22E+08 | minus | 7 |
| Car5b | 1.64E+08 | 1.64E+08 | minus | 8 |
| Car6 | 1.5E+08 | 1.5E+08 | minus | 8 |
| Car7 | 1.05E+08 | 1.05E+08 | plus | 12 |
| Car8 | 8141493 | 8239041 | minus | 9 |
| Car9 | 43507026 | 43513729 | plus | 12 |
| Car10 | 93098141 | 93601751 | plus | 10 |
| Car11 | 45699792 | 45704671 | plus | 6 |
| Car12 | 66711626 | 66766845 | plus | 11 |
| Car13 | 14641727 | 14663002 | plus | 7 |
| Car14 | 95897768 | 95905119 | minus | 12 |
| Car15 | 17835276 | 17638186 | minus | 8 |

Glaucoma

Glaucoma is a condition affecting the eye in which damage occurs to the optic nerve, which can lead to a loss of vision.

Glaucoma may be primary or secondary. (In secondary glaucoma, increased IOP occurs as a result of another condition or injury.)

Sub-types of primary glaucoma include open-angle glaucoma (the most common type), closed-angle glaucoma and normal tension glaucoma (NTG, also known as low tension glaucoma or normal pressure glaucoma).

Secondary glaucoma may result, for example, from eye injury, inflammation (e.g. uveitis), cataracts, conditions that restrict blood flow to the eye such diabetes (diabetic retinopathy), central retinal vein occlusion, neovascularisation (e.g. of the iris, leading to neovascular glaucoma) and tumours.

In all cases, the main cause of damage to the optic nerve is intraocular pressure (IOP). Reduction in intraocular pressure (IOP) largely prevents progression and visual loss by halting continued damage to the optic nerve. This is the case even in normal tension glaucoma, where the IOP is within normal limits (Anderson D R; Normal Tension Glaucoma Study. Collaborative normal tension glaucoma study. Curr. Opin. Ophthalmol. 2003 April; 14(2):86-90).

Currently, the majority of therapeutic approaches to reducing IOP focus on improving drainage (or outflow) of aqueous humour from the eye, for example via the trabecular meshwork or Schlemm's canal. Reducing the production of aqueous humour by the ciliary body has been comparatively neglected, perhaps because suitable approaches have yet to be identified. However, the materials and methods of the present invention provide a simple and straightforward means to inhibit aqueous humour production. As all types of glaucoma can benefit from reducing IOP, or inhibiting its increase, the vectors and methods described herein are believed to have the potential for use in treating any variety of glaucoma, including (but not limited to) those described above.

The materials and methods described in this specification may also be beneficial in conditions causing iris rubeosis (rubeosis iridis) such as neovascular glaucoma, central retinal vein occlusion, ocular ischemic syndrome and chronic retinal detachment. They may also find use in conditions causing “blind painful eye”.

Conditions Associated with CSF Production and Intracranial Pressure

Cerebrospinal fluid (CSF) is produced by the choroid plexus, which is physiologically very similar to the ciliary body. As a result, the vectors and methods described in this specification can be used to treat any condition where inhibiting production of CSF would ameliorate the pathology or symptoms.

Hydrocephalus is a condition in which cerebrospinal fluid (CSF) accumulates within the brain, typically, although not always, leading to raised intracranial pressure. Hydrocephalus may be classified as “communicating” (caused by a defect in reabsorption or drainage to the circulation) or “non-communicating” (caused by a defect in CSF flow within the brain), either of which may be congenital or acquired.

Acquired hydrocephalus may be caused by a wide range of conditions including meningitis, brain tumours and neurological disorders.

Normal pressure hydrocephalus is a specific form of communicating hydrocephalus in which the CSF pressure is within normal boundaries, or only intermittently elevated.

Current treatments involve oral medication, but current drugs show many unacceptable side-effects, limiting their use. Surgical intervention is also possible, by introducing a shunt between brain ventricles and the abdomen, but such procedures have a high rate of failure and complication (up to 48% over 5 years in children and 27% in adults in one study).

Regardless of the underlying cause of the condition, inhibiting the production of CSF should help to reduce the intracranial pressure and so provide therapeutic benefit.

Other relevant conditions which may benefit from inhibiting production of CSF include idiopathic intracranial hypertension (IIH), also known as benign intracranial hypertension (BIH) or pseudotumour cerebri (PTC). Patients with IIH exhibit headache, nausea, vomiting and tinnitus. Left untreated, the condition can cause blindness via swelling of the optic disc.

Pharmaceutical Compositions and Routes of Administration

The nucleic acids, virions, etc. described herein can be formulated in pharmaceutical compositions.

Administration may be peripheral, e.g. intravenous, cutaneous or subcutaneous, nasal, intramuscular or intraperitoneal. Typically, though, administration for treatment of glaucoma will be by intravitreal or intracameral injection and administration for treatment of hydrocephalus will be central, i.e. direct to the central nervous system (CNS), e.g. by intrathecal injection or intracranial injection or infusion, e.g. intracerebroventricular injection or infusion.

Pharmaceutical compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration.

For intravenous, cutaneous or subcutaneous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Compositions for direct administration to the CNS are typically minimal compositions lacking preservatives and other excipients, and may be specially prepared at the time of administration.

Administration is preferably in a “prophylactically effective amount” or a “therapeutically effective amount” (as the case may be), this being sufficient to show benefit to the individual. The actual amount administered, and rate and time-course of administration, may depend on the individual subject and the nature and severity of their condition. Prescription of treatment, e.g. decisions on dosage etc., is within the responsibility of medical practitioners and other medical doctors, and typically takes account of the disorder to be treated, the condition of the individual patient, the site of delivery, the method of administration and other factors known to practitioners.

Examples of the techniques and protocols mentioned above can be found in Remington's Pharmaceutical Sciences, 20th Edition, 2000, pub. Lippincott, Williams & Wilkins.

### EXAMPLES

Materials & Methods

DNA Cloning

Plasmids encoding AAV-SaCas9 with an acceptor site for sgRNA guide insertion was purchased from Addgene (https://www.addgene.org/61591). Using the Golden Gate method, synthesised oligonucleotides (Sigma, UK) were inserted to create different sgRNA guides. Plasmids were expanded using Maxi Prep Plasmid kits (Qiagen, UK).

| Gene | Exon and Name | SaCas9 sgRNA sequence |
|---|---|---|
| Mouse | Exon 1-1B | GATGATGTACATGACAGCCCG |
| Aquaporin 1 | | |
| Mouse | Exon 1-1E | ATCGCTACTCTGGCCCAAAGT |
| Aquaporin 1 | | |

Cell Culture

A spontaneously transformed mouse RPE (Retinal Pigmented Epithelium) cell line B6-RPE07¹ and human Müller cell line (UCLB, London, UK) were cultured in DMEM medium supplemented with 10% heat-inactivated fetal calf serum (FCS), 2 mmol/L L-glutamine, 1 mM Sodium pyruvate, 100 U/mL penicillin and 100 μg/ml streptomycin (all from PAA Laboratories, Pasching, Austria) at 37° C. in an atmosphere of 5% CO₂.

Mice Husbandry

6-8 week old female C57BL/6J mice were used in all in vivo experiments. Mice were obtained from Charles River Laboratories and maintained at the University of Bristol under license from the United Kingdom Home office. In vivo procedures such as fundal imaging with the Micron IV platform (Phoenix research labs, USA) or Tonolab rebound tonometer measurement (iCare, Finland) were performed following anaesthesia with 150 μl of intraperitoneal injection of Ketamine/Rompun mix.

Design of Mouse and Human sgRNA

The mouse Aqp1 cDNA sequence exported from Ensembl (https://www.ensembl.org) was loaded on Benchling (https://benchling.com). Several of the most suitable sgRNAs, based on computationally predicted off-target sites for each target, were selected and cloned into plasmid using Golden Gate Assembly protocol (New England BioLabs). In brief, the selected sgRNA was annealed to be pieces of DNA fragment using T4 PNK enzyme and T4 ligase buffer (New England BioLabs), before being inserted into SaCas9-AAV plasmid using T7 ligase enzyme and T7 ligase buffer (Enzymatics) along with Bsal-HF enzyme (New England BioLabs). The cloned plasmids were next transformed into Competent E. coli (Invitrogen) and cultured in lysogeny broth (LB) to produce large amounts of plasmids for further experiments. Plasmid DNA was extracted by Plasmid miniprep kit (Sigma) or maxiprep kit (Qiagen) following manufacture's protocol. All cloned plasmid DNAs were sequenced (Eurofins Genomics) to check that the sgRNA had been successfully inserted.

Plasmid Transfection In Vitro

Mouse RPE cells and human Müller cells were seeded at a density of 2.5×10⁴/cm² to reach 60-70% cell confluency. On the day of transfection, the culture medium was reduced to half and replaced with plain DMEM for a better efficiency of transfection rate. For each well of cells, 1-2 μg plasmid DNA in 100 ul Opti-MEM (GIBCO, UK) with 0.5-0.75 μl of Lipofectamine 3000 (Invitrogen, UK) was added. Cells were transfected for 48 h before being processed for following experiments.

Adeno-Associated Virus and Intraocular Injections

AAV-CMV-eGFP encoding serotypes AAV 2/1, 2/2, 2/5, 2/6 and 2/8 were purchased from Vector Biolabs, USA. Vectors of the ShH10 serotype were produced at the UCL Institute of Ophthalmology, London and the capsid plasmid was a kind gift of John Flannery, Berkeley University, CA, USA, available on Addgene (https://www.addgene.org/64867). ShH10 virus was produced in HEK-293T cells by triple plasmid transfection before purification using an AVB medium FPLC column (GE life sciences). All viruses were adjusted to a starting concentration of 1×10¹³ genome copies/ml and injected in a volume of 2 μl as intravitreal injection under an operating microscope using a 32-gauge needle and Hamilton syringe.

Immunohistochemistry

Following euthanasia by cervical dislocation, eyes were removed and fixed in cold 4% paraformaldehyde (PFA) for 30 minutes, then placed in optimal cutting temperature embedding medium (Thermo Scientific, UK) and cryosectioned (LEICA, CM3050S) to provide a broad cross-section of each eye, at 12 μm thickness. Eyes were either mounted immediately for GFP detection or stained for immunohistochemical analysis.

Western Blot (WB)

Protein was extracted from cells or tissues by Cellytic MT buffer (Sigma) according to the manufacture's protocol. The concentration of protein was determined using BCA kit (Thermo Fisher Scientific). After 4-12% Bis-Tris Plus gels electrophoresis (Thermo Fisher Scientific), the proteins were transferred to an iBlot PVDF membrane (Thermo Fisher Scientific). After blocking for 1 hour with 5% milk in 0.1% TBST, the membrane was stained with anti-Aqp1 (1:1000, Abcam) in blocking buffer overnight at 4° C. After washing in 0.1% TBST, the membrane was incubated with HRP-conjugated secondary antibody (Cell signalling, MA, USA) or DyLight 800 secondary antibody (Thermo Fisher Scientific). The signals were developed with ECL reagent (Sigma) and captured by an electronic imaging system (Konica Minolta) or Li-Cor imaging system (LI-COR Biosciences). β-actin (Cell signalling) or LaminB1 (Abcam) was used as house-keeping control.

RNA Isolation and Quantitative RT-PCR

Total mRNAs were purified from mouse REP, mouse eye tissues, such as cornea, ciliary body, choroid and retina, using the RNeasy Mini Kit (Qiagen, Hamburg, Germany) as described by the manufacturer's protocol. The one-step TaqMan q-PCR method (AppliedBiosystems) was used to test the Aqp1 gene expression. The results were measured in the same sample and relative ratios of fluorescent intensities of products from each treatment group were calculated by stimuli using the 2^{-ΔΔCt} method.²

GeneArt Genomic Cleavage Assay

Genomic DNA was extracted from transfected cells using DNeasy Blood & Tissue Kit (Qiagen). Loci in which the gene-specific double strand breaks occurred were amplified by PCR using Q5 High-Fidelity DNA polymerase (New England BioLabs) and the following primers Forward: 5′-GGAGGAACTGCTGGCATGCACC-3′; Reverse: 5′-CTAGAGTGCCAGCCTCTGCCCT-3′. The PCR product was denatured and reannealed so that mismatches were generated as strands with an indel reannealed to strands with no indel or a different indel. The mismatches were subsequently detected and cleaved by T7 endonuclease (New England BioLabs) at 37° C. for 20 minutes and terminated by Proteinase K (New England BioLabs) for a 5-minute incubation at 37° C. Fragments were subsequently analysed by agarose gel electrophoresis. For more precise results of cleavage activity, the PCR reactions were sent to University of Bristol Genomics facility for Agilent DNA1000 assay (Agilent Technologies). The results were quantified using ImageJ 1.46r (National Institutes of Health).

In Vivo Experiments

Adult C57BL/6 mice were anesthetized by intraperitoneal (i.p.) injection of Vetelar (ketamine hydrochloride 100 mg/mL, Pfizer, UK) and Rompun (xylazine hydrochloride 20 mg/mL, Bayer, UK) mixed with sterile water in the ratio 0.6:1:84. One eye was intravitreal injected with ShH10-Aqp1 (two plasmids mixed) at volume of 2 μl using a Hamilton microsyringe fitted with a sterile 33-gauge needle. The contralateral eye served as a control was either left untreated or intravitreal injected with ShH10-GFP. The few mice that developed eye abnormalities had been excluded.

IOP was measured by a TonoLab rebound tonometer (TonoVet) when mice were anesthetized using 2.5% isoflurane with 100% oxygen. The IOP was measured in late afternoon (around 4 pm). Mice were sacrificed after either 3 weeks or 6 weeks post treatment for further analysis.

The methods were carried out in accordance with the approved University of Bristol institutional guidelines and all experimental protocols under Home Office Project Licence 30/3045 and 30/3281 were approved by the University of Bristol Ethical Review Group.

Ocular Hypertension Models

Adult C57BL/6 mice were anesthetized with Vetelar and Rompun. Dexamethasone-21-acetate (DEX-Ac) (Sigma, 200 μg/eye) was injected through periocular conjunctival fornix of both eyes for each mouse, following the procedures described before.³ The same DEXA treatment was given once a week to keep IOP stable at a certain level. AAV treatment was randomly injected intravitreally to one eye of each mouse 2 weeks after DEX-Ac treatment.

The induction of microbead occlusion model and preparation of microbeads (Invitrogen) were described before.⁴ IOP baseline was measured before microbeads injection. About 3×10⁶ beads were injected to anterior chamber of each eye after pupil was dilated by tropicamide eye drop. AAV treatment was randomly injected intravitreally to one eye of each mouse 1 week after microbeads injection.

After 3 weeks post AAV treatment, all mice received IOP measurement. The thickness of retina and cornea was measured at the end point of experiment using Micron IV (Phoenix research labs). Mice were then sacrificed for Aqp1 protein expression in ciliary body and ganglion cell count in retina.

Immunofluorescence and Immunohistochemistry

Mouse eyes were dissected and fixed in 2% paraformaldehyde (Thermo Fisher) over night at 4° C. followed by dehydrating with ethanol before being sent to Histology Lab in University of Bristol for paraffin embedding and hematoxylin and eosin (H&E) staining.

Mice eyes were snap frozen in OCT (optical cutting temperature compound, Thermo, Waltham, Mass., USA) using dry ice. Cryosections of 12 μm thickness were fixed in 4% PFA for 10 minutes and immunostained with rabbit anti-mouse Aqp1 (at dilution 1:100, Abcam), and goat anti-mouse CD45 (at dilution 1:100, BD Biosciences) antibodies in conjunction with the secondary antibodies (at dilution 1:200, Life Technologies). Mice retina was dissected and fixed in 4% PFA for 2 hours. After an hour blocking in 2% Trition and 2% BSA, the retina was stained with Brn-3a (at dilution of 1:50, SantaCruz) over night before flat mount. All images were taken under Leica SP5-AOBS confocal laser scanning microscope. The ganglion cell counting of Brn-3a was analysed by Volocity (Version 6.2.1).

Brominated Thymidine Analog 5-Bromo-2′-Deosyuridine (BrdU) Assay

BrdU solution (BD Biosciences) was injected to each mouse through i.p. with 100 ul (1 μg). After 24 h, mice were sacrificed for eye cryo-sections. Frozen sections were fixed by 4% PFA for 10 minutes. After washing with PBS, the sections were incubated with 2N HCL at 37° C. for 15 minutes. Followed by PBS washing, the sections were then incubated with anti-BudU biotin antibody (eBioscience) for 2 hours at room temperature, followed by an incubation of streptavidin for BrdU eFluor 570 (eBioscience). Finally, the sections were mounted for Confocal imaging (Leica SP5-AOBS).

Human Tissue Culture and Transfection

Human eye tissues were isolated from normal donor eyes. All donor eyes were obtained from Bristol Eye Bank after receipt of informed consent from the donor's families and were managed according to the guidelines in the Declaration of Helsinki on research involving human tissue. Isolated human ciliary body were cultured in epithelial cell culture medium (ScienCell Research Laboratories). The ciliary body was treated with ShH10-GFP or ShH10-Aqp1 for a time course incubation. Ciliary body tissue was collected for cryosection to detect GFP expression at 24 h-, 72 h- and 7-day culture. The cleavage activity and Aqp1 protein expression were also detected and quantified in treated ciliary body tissue at the end time point.

Statistics

Results are therefore presented as means±standard deviation (S.D.). Comparisons of two individual experimental groups were performed by unpaired Student's t test and Mann-Whitney test. For multiple comparisons, nonparametric analysis was performed using one-way ANOVA test with Dunn's test. All the analysis was performed using GraphPad Prism 6 (GraphPad Software, version 6.01, La Jolla, USA). Two-tailed tests were used throughout. The significant differences were considered at P≤0.05.
1 Chen, M. et al. Characterization of a spontaneous mouse retinal pigment epithelial cell line B6-RPE07. Investigative ophthalmology & visual science 49, 3699-3706, doi:10.1167/iovs.07-1522 (2008).
2 Livak, K. J. & Schmittgen, T. D. Analysis of relative gene expression data using real-time quantitative PCR and the 2(-Delta Delta C(T)) Method. Methods (San Diego, Calif.) 25, 402-408, doi:10.1006/meth.2001.1262 (2001).
3 Patel, G. C. et al. Dexamethasone-Induced Ocular Hypertension in Mice: Effects of Myocilin and Route of Administration. The American journal of pathology 187, 713-723, doi:10.1016/j.ajpath.2016.12.003 (2017).
4 Ito, Y. A., Belforte, N., Cueva Vargas, J. L. & Di Polo, A. A Magnetic Microbead Occlusion Model to Induce Ocular Hypertension-Dependent Glaucoma in Mice. Journal of visualized experiments: JoVE, e53731, doi:10.3791/53731 (2016).

Results

AAV permits permanent expression in post-mitotic cells, and as there is almost no turnover of ciliary body epithelium, gene delivery to these cells should endure. We have confirmed that intravitreal injection of the ShH10 serotype in the mouse results in efficient ciliary body transduction and is the only tested serotype capable of infecting the ciliary body from the intravitreal route (Table 1). Off-target ocular tissues were also characterized. Three weeks after intravitreal injection of 2 μl of ShH10-CMV-eGFP at titres of 5×10¹³, 5×10¹² and 5×10¹¹genome copies/ml, eyes were examined by frozen section for the presence of GFP signal. GFP expression was observed in ciliary body epithelial cells, while levels of retinal transduction fell as the titre used for administration was reduced (data not shown). We also identified that ShH10 can infect the human ciliary body epithelium using ex vivo tissue from donor eyes. The scleral ring was dissected from a donor eye obtained within 24 hours after death and maintained in culture for 48 hours before addition of vehicle or 2 μl of ShH10-CMV-eGFP at 1×10¹³genome copies/ml. Fluorescent microscopy after 48 hours of culture showed no GFP signal in cultures without virus, whereas those treated with 1×10¹³ gc ShH10-CMV-eGFP showed widespread low level signal and focal points of strong signal at edge regions (data not shown).

Subsequent experiments have shown that good levels of ciliary body transduction, with very little Muller glial cell infection, can be achieved with a 2 μl bolus at a concentration of 1×10¹¹ gc/ml.

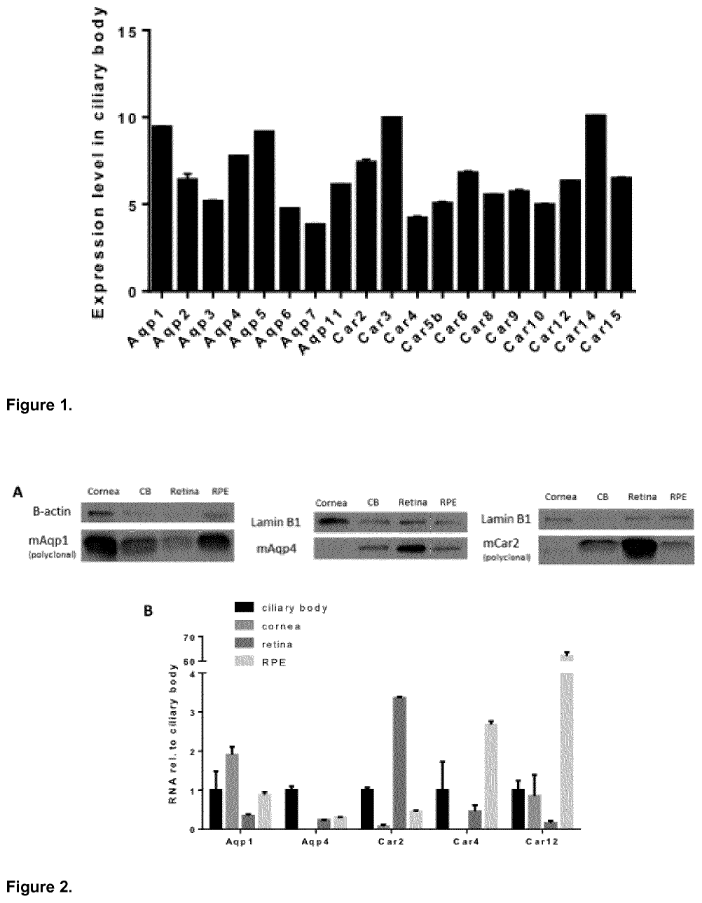

Analyzing microarray databases, we constructed a relative expression map of Aquaporin and Carbonic anhydrase isoforms in the mouse eye. This identified Aqp1, 4, 5 and Car2, 3 and 14 as the most abundant transcripts and therefore likely to play a key role in aqueous humour production (FIG. 1). The protein and RNA expression and tissue distribution of key Aquaporin and Carbonic anhydrase isoforms was determined for the mouse eye (FIG. 2). It confirmed ciliary body expression and guides selection of gene targets.

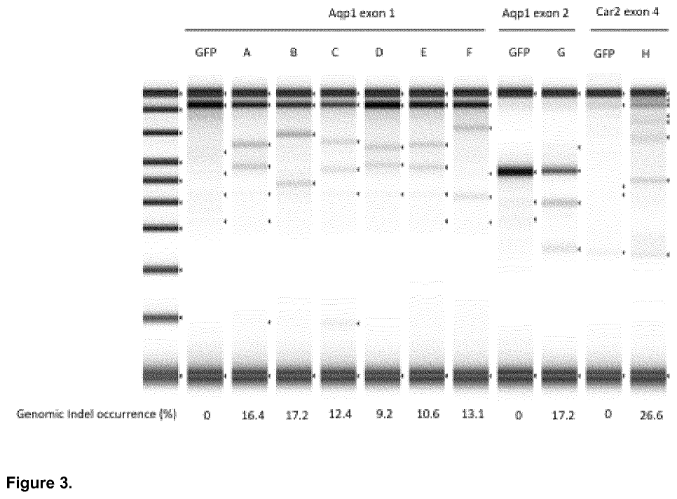

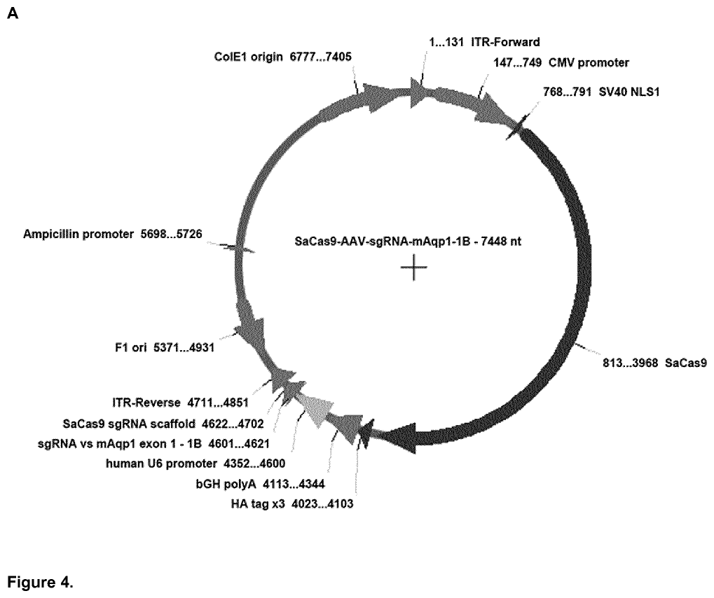

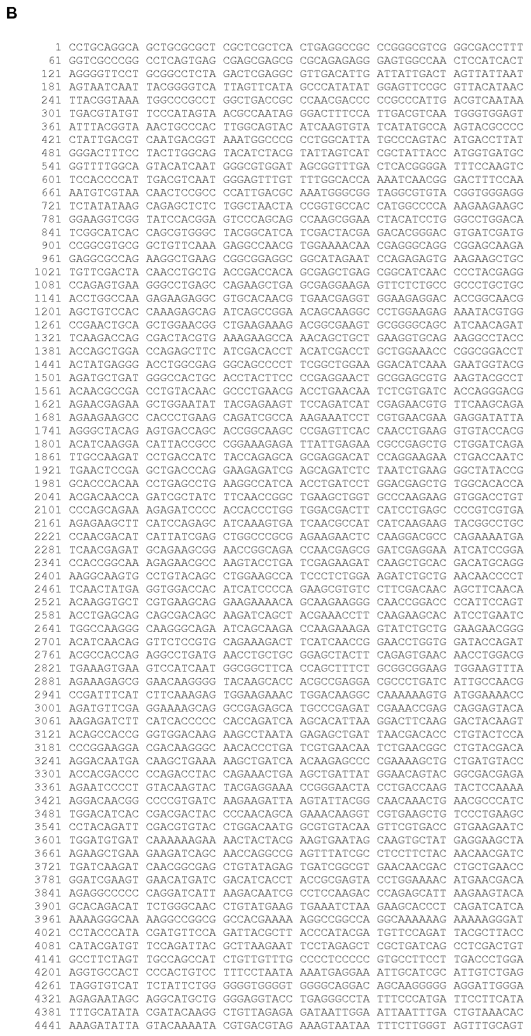

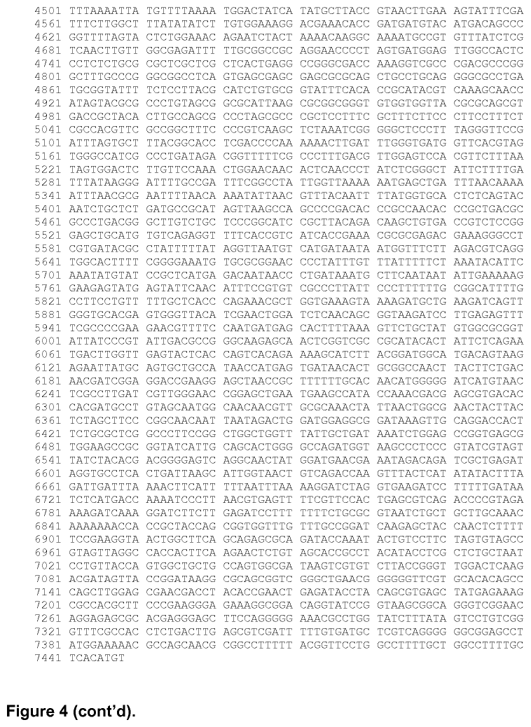

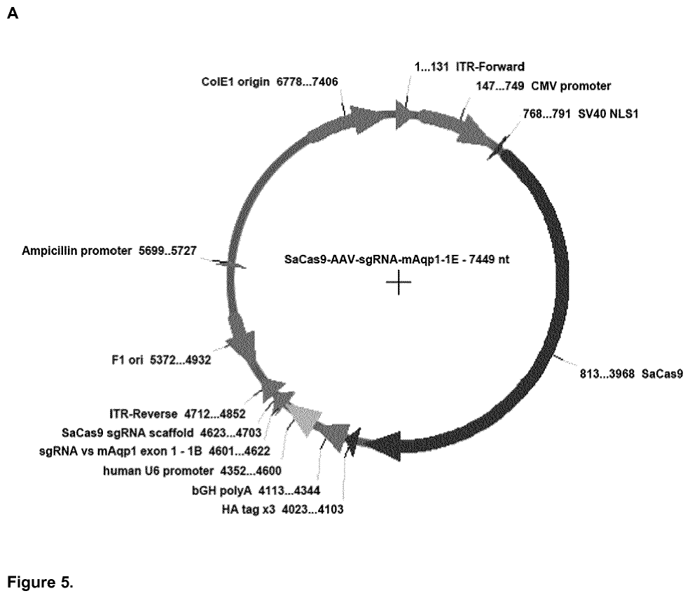

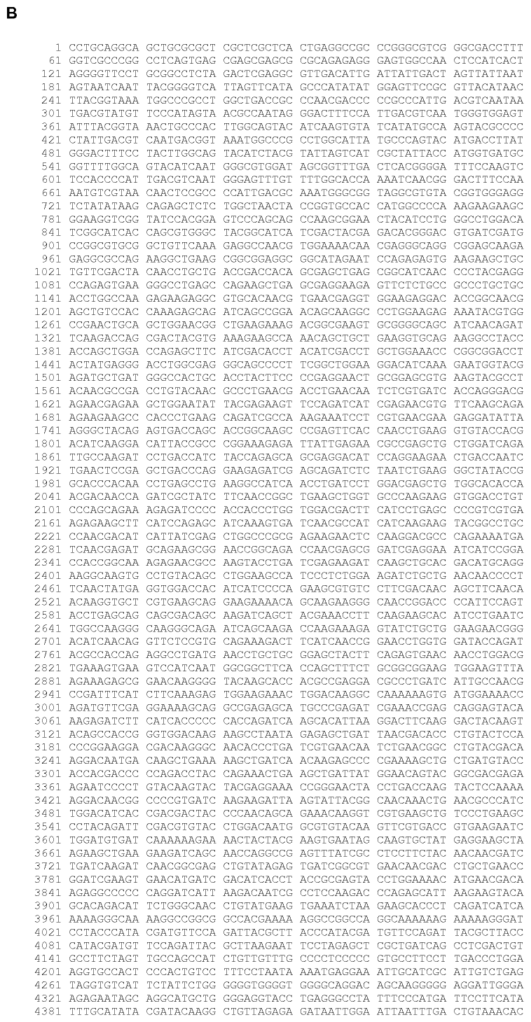

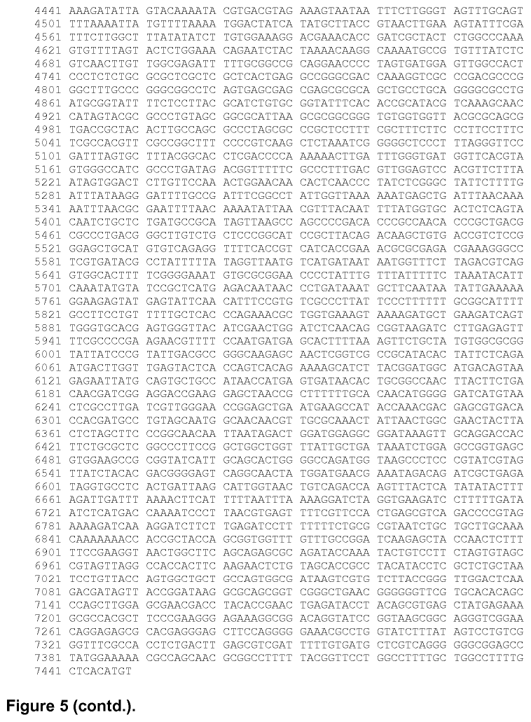

The CRISPR-Cas9 system using the newly described SaCas9 which is capable of being packaged into AAV was tested. This system uses RNA guides (sgRNA) to direct SaCas9 to cause a double-strand DNA break into a target gene, typically leading to indel formation and a premature stop codon. Several SaCas9 guide RNA were tested in vitro with a mouse ocular cell line targeting Aqp1 and Car2. T7 endonuclease I cleavage assay identified several active guide RNAs with indel efficiencies of up to 26% (FIG. 3). Two promising guide RNAs were produced and packaged into ShH10 vectors. Plasmid maps and sequences are show in FIGS. 4 and 5.

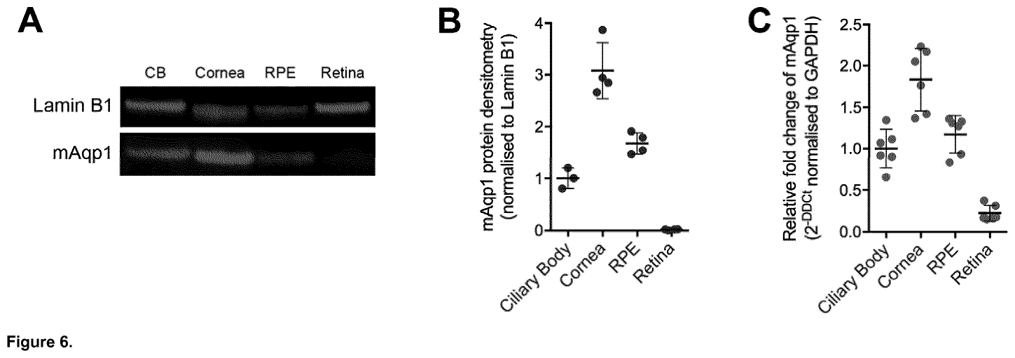

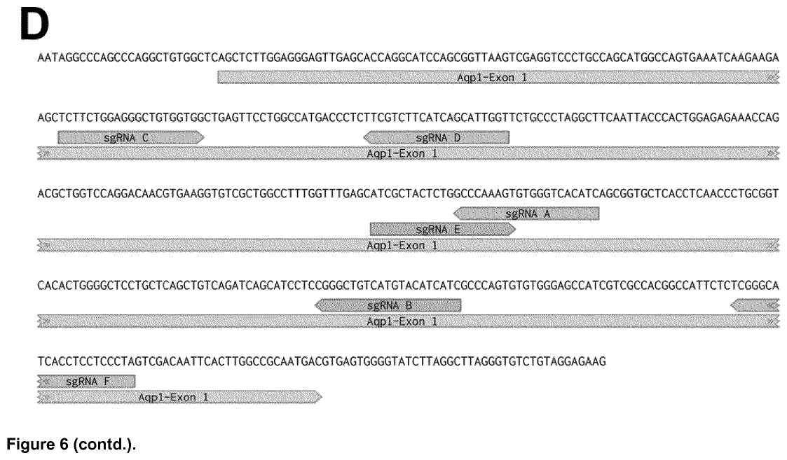

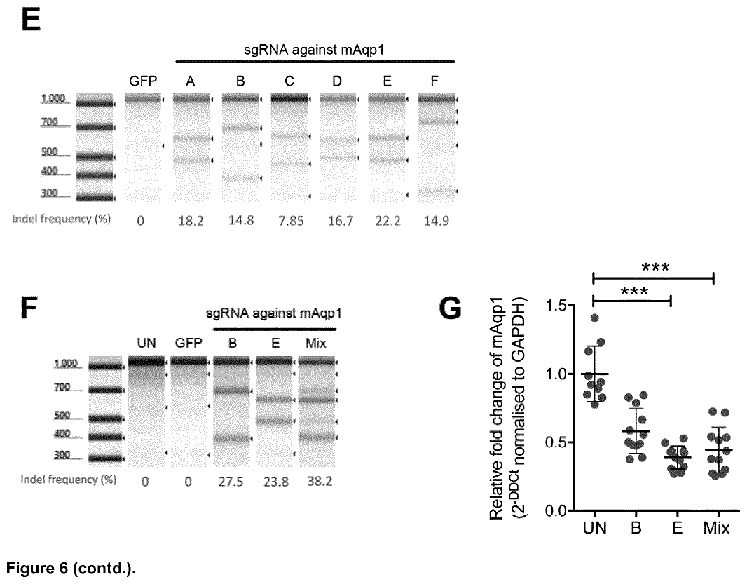

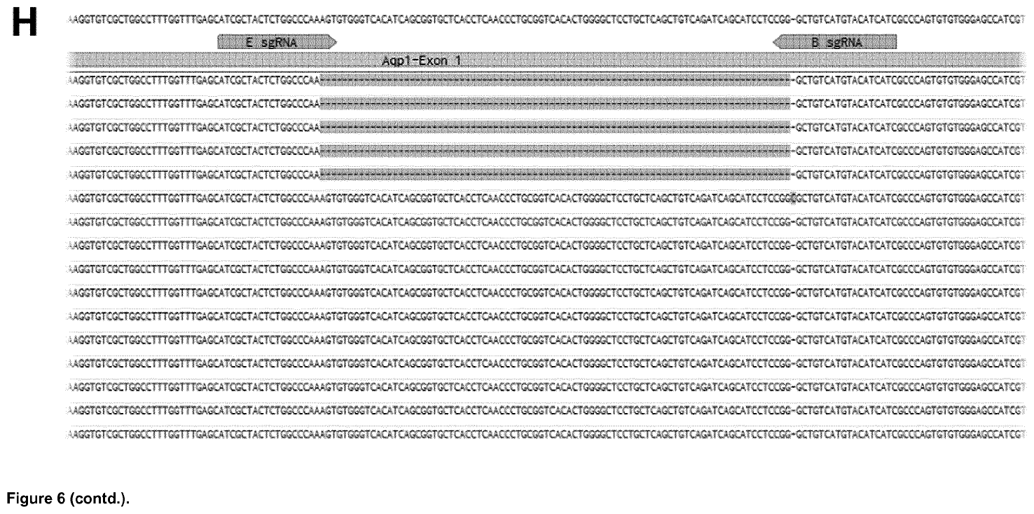

Aqp1 is enriched in mouse ciliary body though is also present in the cornea. Levels were characterised by quantitative PCR and Western blot (FIG. 6A-C). Several SaCas9 specific sgRNA were designed targeting exon 1 of mouse Aqp1 and tested on a mouse B6-RPE cell line for efficacy using T7 endonuclease 1 assay Two Aqp1 sgRNAs (B and E, also known as 1B and 1E) were selected to be packaged into vectors due to their better efficiency of disrupting Aqp1 transcription in B6-RPE cells than other sgRNAs and optimum spacing across exon 1. A mixture of the two vectors was used to infect B6-RPE cells in vitro and disruption of Aqp1 RNA transcripts was confirmed after 72 hours compared to untreated or GFP virus infected cells (FIG. 6F). Disruption of Aqp1 RNA transcript was also confirmed using a 50:50 mix of virus encoding SaCas9 and sgRNA 1B and 1E (referred to as ‘Mix’) in the B6-RPE line (FIG. 6G). On-target effects including complete excision of the intervening exon1 region was confirmed (FIG. 6H).

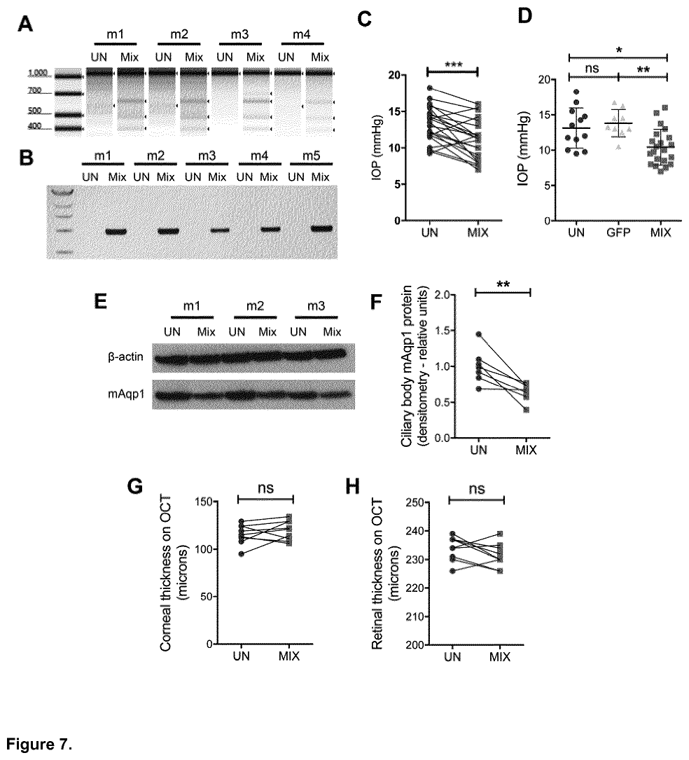

The same mixture of two ShH10 vectors (Mix′) targeting exon 1 of Aqp1 was injected into the vitreous cavity of wildtype C57BL/6J mice. Three weeks after injection the ciliary body was dissected from selected eyes and both genomic editing of the Aqp1 locus, the presence of SaCas9 DNA and reduced IOP by a mean of 2.9 mmHg was observed (FIG. 7C). No reduction in IOP was observed using control GFP expressing ShH10 virus. Ciliary body Aqp1 protein levels were tested for Aqp1 by Western blot (FIG. 7E-F). Compared to control eyes, the level of Aqp1 was reduced in CRISPR treated eyes. Complete disruption would not be seen as it is not feasible to dissect away only viral transduced non-pigmented ciliary body epithelium for Western blotting. Off-target effects including corneal oedema and thickening or retinal oedema were not seen using in vivo OCT imaging (FIG. 7G-H).

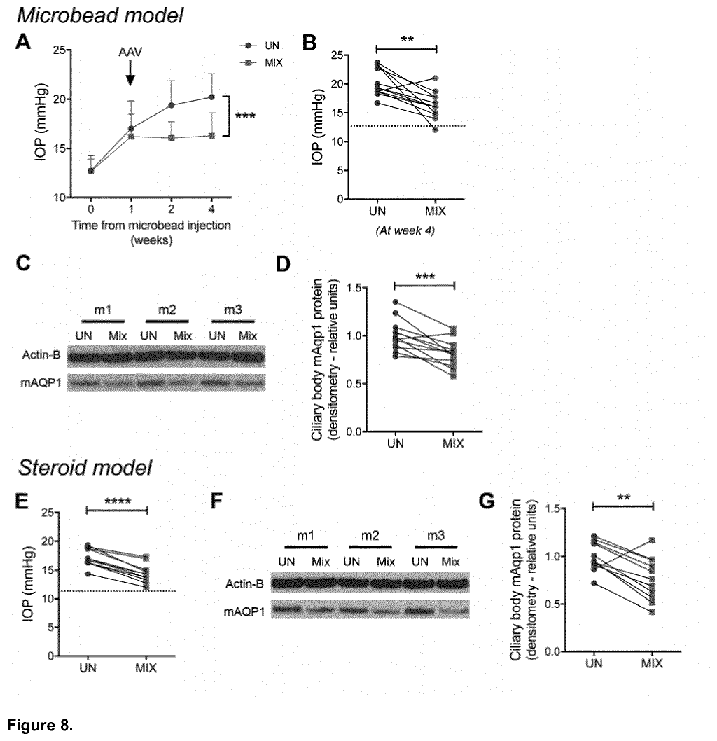

The same mixture of ShH10-SaCas9 vectors was introduced into one eye of two ocular hypertension mice models. Three weeks after vectors treatment, there was a significant reduction of IOP and Aqp1 protein expression in ciliary body in both models. The average IOP reduction was 3.9 mmHg and 2.9 mmHg in microbeads and steroid model respectively (FIG. 8).

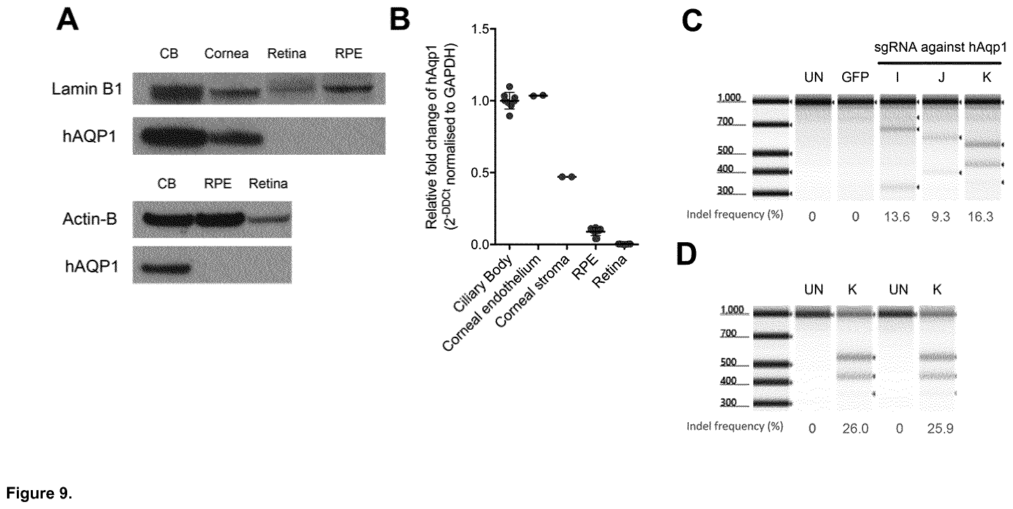

Using ex vivo donor human eyes, Aqp1 is also detected and enriched in human ciliary body by Western blotting and quantitative PCR (FIG. 9A-B). Therefore, several human sgRNA guides were designed and tested on the human 293T cell line. One sgRNA K of human Aqp1 was selected out of a number of sgRNAs as the most efficacious, which was further packaged into the ShH10 vector and then tested on 293T cells, which confirmed genomic editing in the human Aqp1 locus (FIG. 9D). We have also shown using ShH10 virus encoding GFP under the control of the ubiquitous CMV promoter that ShH10 is capable of infecting and transducing human ciliary body by co-culture for up to 7 days (data not shown).

TABLE 1
ShH10 is the only AAV serotype tested capable of ciliary body
epithelium transduction following intravitreal injection. C57BL/6J
mice underwent intravitreal injection with 2 μl of 1 × 10¹³ gc/ml of
different AAV serotypes expressing eGFP under the CMV promoter.
After three weeks eyes were enucleated, cryo-sectioned at 16 μm
thickness, mounted and imaged by confocal microscopy. Five
independent eyes injected with each serotype
were examined, with the summary of results displayed.

| AAV | Target Tissue | | | |
|---|---|---|---|---|
| Serotype | Ciliary Body | Retina | Cornea | RPE |
| AAV 2/1 | ND | ND | ND | ND |
| AAV 2/2 | ND | Detected | ND | ND |
| AAV 2/5 | ND | ND | ND | ND |
| AAV 2/6 | ND | Detected | ND | ND |
| AAV 2/8 | ND | Detected | Detected | ND |
| ShH10 | Detected | Detected | Detected | ND |

(ND = Not detected).

Further investigations were carried out to investigate the suitability of our approach for modulating cerebrospinal fluid formation in the choroid plexus, e.g. as a therapy for hydrocephalus.

Choroid plexus from the lateral ventricle of C57BL/6J mice was dissected out and placed into tissue culture. 5×10¹¹ genome copies of ShH10 encoding CMV-GFP were added and incubated for 72 hours. GFP expression was seen along the choroid plexus epithelium, demonstrating that ShH10 has the capacity to infect these cell types. (Data not shown.) Immunohistochemistry on coronal cryo-sections of C57BL/6J mouse brain demonstrated the presence of Aqp1 and Aqp4 in the choroid plexus. Aqp1 expression appeared to be restricted to the choroid plexus only, whilst Aqp4 was present in cortical neurons. (Data not shown.)

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All documents cited herein are expressly incorporated by reference.

The teaching of all references in the present application, including patent applications and granted patents, are herein fully incorporated by reference. Any patent application to which this application claims priority is incorporated by reference herein in its entirety in the manner described herein for publications and references.

For the avoidance of doubt the terms ‘comprising’, ‘comprise’ and ‘comprises’ herein is intended by the inventors to be optionally substitutable with the terms ‘consisting of’, ‘consist of’, and ‘consists of’, respectively, in every instance. The term “about” (or “around”) in all numerical values allows for a 5% variation, i.e. a value of about 1.25% would mean from between 1.19%-1.31%.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the measurement, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. An AAV vector virion of serotype ShH10, comprising:
(i) a nucleic acid sequence encoding an RNA-guided endonuclease; and
(ii) a nucleic acid sequence encoding a guide RNA complementary to a target sequence from an aquaporin gene or a carbonic anhydrase gene and capable of directing said RNA-guided endonuclease to said target sequence.

2. An AAV vector virion for use in a method of modulating intraocular pressure or production of aqueous humour, wherein the AAV vector is of serotype ShH10 and comprises:
(i) a nucleic acid sequence encoding an RNA-guided endonuclease; and
(ii) a nucleic acid sequence encoding a guide RNA complementary to a target sequence from an aquaporin gene or a carbonic anhydrase gene and capable of directing said RNA-guided endonuclease to said target sequence.

3. An AAV vector virion for use according to claim 2 wherein said use is use in the treatment of ocular hypertension and/or glaucoma.

4. An AAV vector virion for use according to claim 3 wherein said glaucoma is primary or secondary glaucoma.

5. An AAV vector virion for use according to claim 3 or claim 4 wherein said primary glaucoma is open-angle glaucoma, closed-angle glaucoma or normal tension glaucoma (NTG).

6. An AAV vector virion, or an AAV vector virion for use, according to any one of the preceding claims, wherein the aquaporin (AQP) gene is AQP1, AQP2, AQP3, AQP4, AQP5, AQP6, AQP7 or AQP11.

7. An AAV vector virion, or AAV vector virion for use, according to claim 6, wherein the aquaporin gene is AQP1, AQP4 or AQP5.

8. An AAV vector virion, or an MV vector virion for use, according to any of the preceding claims, wherein the carbonic anhydrase (CAR) gene is CAR2, CAR3, CAR4, CR5b, CARE, CARE, CAR9, CAR10, CAR12 or CAR14.

9. An AAV vector virion, or an AAV vector virion for use, according to claim 8, wherein the CAR gene is CAR2, CAR3, CAR4, CAR12 or CAR14.

10. An AAV vector virion for use in a method of modulating intracranial pressure or production of CSF, wherein the AAV vector is of serotype ShH10 and comprises:
(i) a nucleic acid sequence encoding an RNA-guided endonuclease; and
(ii) a nucleic acid sequence encoding a guide RNA complementary to a target sequence from an aquaporin gene or a carbonic anhydrase gene and capable of directing said RNA-guided endonuclease to said target sequence.

11. An AAV vector virion for use according to claim 10 wherein said use is use in the treatment of hydrocephalus or idiopathic intracranial hypertension.

12. An AAV vector virion for use according to claim 11 wherein said hydrocephalus is communicating hydrocephalus or non-communicating hydrocephalus.

13. An AAV vector virion for use according to claim 11 or claim 12 wherein said hydrocephalus is normal pressure hydrocephalus.

14. An AAV vector virion for use according to any one of claims 11 to 13 wherein said hydrocephalus is congenital or acquired.

15. An AAV vector virion according to claim 1 or an AAV vector virion for use according to any of claims 10 to 14, wherein the aquaporin (AQP) gene is AQP1, AQP2, AQP3, AQP4, AQP5, AQP6, AQP7 or AQP11.

16. An AAV vector virion, or an AAV vector virion for use, according to claim 15, wherein the aquaporin gene is AQP1 or AQP4.

17. An AAV vector virion, or an AAV vector virion for use, according to any of claim 1 or 10 to 16, wherein the carbonic anhydrase (CAR) gene is CAR2, CAR3, CAR4, CR5b, CAR6, CAR8, CAR9, CAR10, CAR12 or CAR14.

18. An AAV vector virion, or an AAV vector virion for use, according to claim 17, wherein the CAR gene is CAR2, CAR3, CAR4, CAR12 or CAR14.

19. An AAV vector virion, or an MV vector virion for use, according to any of the preceding claims, wherein the RNA-guided endonuclease is a Cas9 enzyme.

20. An AAV vector virion, or an AAV vector virion for use, according to claim 19, wherein the Cas9 enzyme is Staphylococcus aureus Cas9 (SaCas9), Streptococcus pyogenes Cas9 (SpCas9), Neisseria meningitidis Cas9 (NM Cas9), Streptococcus thermophilus Cas9 (ST Cas9), Treponema denticola Cas9 (TD Cas9), or a variant thereof.

21. An AAV vector virion, or an AAV vector virion for use, according to claim 20, wherein the variant is SpCas9 D1135E, SpCas9 VRER, SpCas9 EQR or SpCas9 VQR.

22. An AAV vector virion, or an AAV vector virion for use, according to any of the preceding claims, wherein the RNA-guided endonuclease is catalytically active.

23. An AAV vector virion, or an AAV vector virion for use, according to any of claims 1 to 22, wherein the RNA-guided endonuclease is catalytically dead and further comprises a transcriptional repressor domain.

24. An AAV vector virion, or an AAV vector virion for use, according to claim 23, wherein the transcriptional repressor domain is a Kruppel associated box (KRAB) domain, CS domain, WRPW domain, MXI1, mSin3 interacting domain, or histone demethylase LSD1 domain.

25. An AAV vector virion, or an AAV vector virion for use, according to any of the preceding claims, wherein the endonuclease further comprises a nuclear localisation sequence effective in mammalian cells.

26. A pharmaceutical composition comprising an AAV vector virion as defined in any one of the preceding claims, in combination with pharmaceutically acceptable carrier.

27. A pharmaceutical composition according to claim 26, formulated for intraocular injection.

28. A pharmaceutical composition according to claim 26, formulated for intravitreal or intracameral injection.

29. A pharmaceutical composition according to claim 26, formulated for central administration.

30. A pharmaceutical composition according to claim 29, formulated for intrathecal injection, intracranial injection, intracranial infusion, intracerebroventricular injection, or intracerebroventricular infusion.

31. A packaging cell, producing an AAV vector virion as defined in any one of claims 1 to 25.

32. A therapeutic kit comprising first and second AAV vector virions as defined in any one of claims 1 to 25, said first and second AAV vector virions encoding respective different first and second guide RNAs complementary to respective different first and second target sequences.

33. A therapeutic kit according to claim 32, wherein the first and second target sequences are from the same aquaporin or carbonic anhydrase gene.

34. A therapeutic kit according to claim 32 or claim 33 wherein the first and second vector virions are otherwise identical apart from the encoded guide RNAs.

35. A therapeutic kit according to any one of claims 32 to 34 wherein said first and second vector virions are formulated in separate compositions, each in combination with a pharmaceutically acceptable carrier.

36. A therapeutic kit according to any one of claims 32 to 34 wherein said first and second vector virions are formulated in the same composition, in combination with a pharmaceutically acceptable carrier.

37. A therapeutic kit comprising:
(a) a first AAV vector virion of serotype ShH10 comprising:
(i) a nucleic acid sequence encoding an RNA-guided endonuclease; and
(ii) a nucleic acid sequence encoding a first guide RNA complementary to a first target sequence from an aquaporin gene or a carbonic anhydrase gene and capable of directing said RNA-guided endonuclease to first said target sequence; and
(b) a second AAV vector virion of serotype ShH10, comprising:
(i) a nucleic acid sequence encoding an RNA-guided endonuclease; and
(ii) a nucleic acid sequence encoding a second guide RNA complementary to a second target sequence from an aquaporin gene or a carbonic anhydrase gene and capable of directing said RNA-guided endonuclease to said second target sequence.

38. A kit according to claim 37, wherein the first and/or second target sequence is from an aquaporin (AQP) gene.

39. A kit according to claim 38, wherein the aquaporin (AQP) gene is AQP1, AQP2, AQP3, AQP4, AQP5, AQP6, AQP7 or AQP11.

40. A kit according to claim 39, wherein the aquaporin gene is AQP1, AQP4 or AQP5.

41. A kit according to any one of claims 37 to 40, wherein the first and/or second target sequence is from a carbonic anhydrase (CAR) gene.

42. A kit according to claim 41, wherein the carbonic anhydrase (CAR) gene is CAR2, CAR3, CAR4, CR5b, CARE, CARE, CAR9, CAR10, CAR12 or CAR14.

43. A kit according to claim 43, wherein the CAR gene is CAR2, CAR3, CAR4, CAR12 or CAR14.

44. A kit according to any one of claims 37 to 43, wherein the or each RNA-guided endonuclease is a Cas9 enzyme.

45. A kit according to claim 44, wherein the Cas9 enzyme is Staphylococcus aureus Cas9 (SaCas9), Streptococcus pyogenes Cas9 (SpCas9), Neisseria meningitidis Cas9 (NM Cas9), Streptococcus thermophilus Cas9 (ST Cas9), Treponema denticola Cas9 (TD Cas9), or a variant thereof.

46. A kit according to claim 45, wherein the variant is SpCas9 D1135E, SpCas9 VRER, SpCas9 EQR or SpCas9 VQR.

47. A kit according to any one of claims 37 to 46, wherein the or each RNA-guided endonuclease is catalytically active.

48. A kit according to any one of claims 37 to 46, wherein the or each RNA-guided endonuclease is catalytically dead and further comprises a transcriptional repressor domain.

49. A kit according to claim 48, wherein the transcriptional repressor domain is a Kruppel associated box (KRAB) domain, CS domain, WRPW domain, MXI1, mSin3 interacting domain, or histone demethylase LSD1 domain.

50. A kit according to any one of claims 37 to 49, wherein the or each endonuclease further comprises a nuclear localisation sequence effective in mammalian cells.

51. A kit according to any one of claims 37 to 50 wherein the first and second target sequences are from the same gene.

52. An AAV vector virion for use in a method of modulating intraocular pressure or production of aqueous humour, wherein the AAV vector virion is of serotype ShH10 and comprises:
(i) a nucleic acid sequence encoding an RNA-guided endonuclease; and
(ii) a nucleic acid sequence encoding a first guide RNA complementary to a first target sequence from an aquaporin gene or a carbonic anhydrase gene and capable of directing said RNA-guided endonuclease to first said target sequence;
wherein the AAV vector virion is for administration in combination with a second AAV vector virion of serotype ShH10, comprising:
(i) a nucleic acid sequence encoding an RNA-guided endonuclease; and
(ii) a nucleic acid sequence encoding a second guide RNA complementary to a second target sequence from an aquaporin gene or a carbonic anhydrase gene and capable of directing said RNA-guided endonuclease to said second target sequence.

53. An AAV vector virion for use according to claim 52 wherein said use is use in the treatment of ocular hypertension and/or glaucoma.

54. An AAV vector virion for use according to claim 53 wherein said glaucoma is primary or secondary glaucoma.

55. An AAV vector virion for use according to claim 53 or claim 54 wherein said primary glaucoma is open-angle glaucoma, closed-angle glaucoma or normal tension glaucoma (NTG).

56. An AAV vector virion for use according to any one of claims 52 to 55, wherein the first and/or second target sequence is from an aquaporin (AQP) gene.

57. An AAV vector virion for use according to claim 56, wherein the aquaporin gene is AQP1, AQP2, AQP3, AQP4, AQP5, AQP6, AQP7 or AQP11.

58. An AAV vector virion for use according to claim 57, wherein the aquaporin gene is AQP1, AQP4 or AQP5.

59. An AAV vector virion for use according to any one of claims 52 to 58, wherein the first and/or second target sequence is from a carbonic anhydrase (CAR) gene.

60. An MV vector virion for use according to claim 59 wherein the CAR gene is CAR2, CAR3, CAR4, CR5b, CAR6, CAR8, CAR9, CAR10, CAR12 or CAR14.

61. An AAV vector virion for use according to claim 60, wherein the CAR gene is CAR2, CAR3, CAR4, CAR12 or CAR14.

62. An AAV vector virion for use in a method of modulating intracranial pressure or production of CSF, wherein the AAV vector virion is of serotype ShH10 and comprises:
(i) a nucleic acid sequence encoding an RNA-guided endonuclease; and
(ii) a nucleic acid sequence encoding a first guide RNA complementary to a first target sequence from an aquaporin gene or a carbonic anhydrase gene and capable of directing said RNA-guided endonuclease to first said target sequence;
wherein the AAV vector virion is for administration in combination with a second AAV vector virion of serotype ShH10, comprising:
(i) a nucleic acid sequence encoding an RNA-guided endonuclease; and
(ii) a nucleic acid sequence encoding a second guide RNA complementary to a second target sequence from an aquaporin gene or a carbonic anhydrase gene and capable of directing said RNA-guided endonuclease to said second target sequence.

63. An AAV vector virion for use according to claim 62 wherein said use is use in the treatment of hydrocephalus or idiopathic intracranial hypertension.

64. An AAV vector virion for use according to claim 63 wherein said hydrocephalus is communicating hydrocephalus or non-communicating hydrocephalus.

65. An AAV vector virion for use according to claim 63 or claim 64 wherein said hydrocephalus is normal pressure hydrocephalus.

66. An AAV vector virion for use according to any one of claims 62 to 65 wherein said hydrocephalus is congenital or acquired.

67. An AAV vector virion for use according to any one of claims 62 to 66, wherein the first and/or second target sequence is from an aquaporin (AQP) gene.

68. An AAV vector virion for use according to any one of claims 62 to 67, wherein the aquaporin (AQP) gene is AQP1, AQP2, AQP3, AQP4, AQP5, AQP6, AQP7 or AQP11.

69. An AAV vector virion for use according to claim 68, wherein the aquaporin gene is AQP1 or AQP4.

70. An AAV vector virion for use according to any one of claims 62 to 69, wherein the first and/or second target sequence is from a carbonic anhydrase (CAR) gene.

71. An AAV vector virion for use according to claim 70, wherein the carbonic anhydrase (CAR) gene is CAR2, CAR3, CAR4, CR5b, CAR6, CAR8, CAR9, CAR10, CAR12 or CAR14.

72. An AAV vector virion for use according to claim 71, wherein the CAR gene is CAR2, CAR3, CAR4, CAR12 or CAR14.

73. An AAV vector virion for use according to any one of claims 52 to 72, wherein the or each RNA-guided endonuclease is a Cas9 enzyme.

74. An AAV vector virion for use according to claim 73, wherein the Cas9 enzyme is Staphylococcus aureus Cas9 (SaCas9), Streptococcus pyogenes Cas9 (SpCas9), Neisseria meningitidis Cas9 (NM Cas9), Streptococcus thermophilus Cas9 (ST Cas9), Treponema denticola Cas9 (TD Cas9), or a variant thereof.

75. An AAV vector virion for use according to claim 74, wherein the variant is SpCas9 D1135E, SpCas9 VRER, SpCas9 EQR or SpCas9 VQR.

76. An AAV vector virion for use according to any one of claims 52 to 75, wherein the or each RNA-guided endonuclease is catalytically active.

77. An AAV vector virion for use according to any one of claims 52 to 75, wherein the or each RNA-guided endonuclease is catalytically dead and further comprises a transcriptional repressor domain.

78. A kit according to claim 77, wherein the transcriptional repressor domain is a Kruppel associated box (KRAB) domain, CS domain, WRPW domain, MXI1, mSin3 interacting domain, or histone demethylase LSD1 domain.

79. A kit according to any one of claims 52 to 78, wherein the or each endonuclease further comprises a nuclear localisation sequence effective in mammalian cells.

80. A kit according to any one of claims 52 to 79, wherein the first and second target sequences are from the same gene.

81. A therapeutic kit comprising:
(a) a first AAV vector virion of serotype ShH10, comprising a nucleic acid sequence encoding an RNA-guided endonuclease; and
(b) a second AAV vector virion of serotype ShH10, comprising a nucleic acid sequence encoding a guide RNA complementary to a target sequence from an aquaporin gene or a carbonic anhydrase gene and capable of directing said RNA-guided endonuclease to said target sequence.

82. A kit according to claim 81, wherein the aquaporin (AQP) gene is AQP1, AQP2, AQP3, AQP4, AQP5, AQP6, AQP7 or AQP11.

83. A kit according to claim 82, wherein the aquaporin gene is AQP1, AQP4 or AQP5.

84. A kit according to claim 81, wherein the carbonic anhydrase (CAR) gene is CAR2, CAR3, CAR4, CR5b, CARE, CARE, CAR9, CAR10, CAR12 or CAR14.

85. A kit according to claim 84, wherein the CAR gene is CAR2, CAR3, CAR4, CAR12 or CAR14.

86. A kit according to any one of claims 81 to 85, wherein the RNA-guided endonuclease is a Cas9 enzyme.

87. A kit according to claim 86, wherein the Cas9 enzyme is Staphylococcus aureus Cas9 (SaCas9), Streptococcus pyogenes Cas9 (SpCas9), Neisseria meningitidis Cas9 (NM Cas9), Streptococcus thermophilus Cas9 (ST Cas9), Treponema denticola Cas9 (TD Cas9), or a variant thereof.

88. A kit according to claim 87, wherein the variant is SpCas9 D1135E, SpCas9 VRER, SpCas9 EQR or SpCas9 VQR.

89. A kit according to any one of claims 81 to 89, wherein the or each RNA-guided endonuclease is catalytically active.

90. A kit according to any one of claims 81 to 89, wherein the or each RNA-guided endonuclease is catalytically dead and further comprises a transcriptional repressor domain.

91. A kit according to claim 90, wherein the transcriptional repressor domain is a Kruppel associated box (KRAB) domain, CS domain, WRPW domain, MXI1, mSin3 interacting domain, or histone demethylase LSD1 domain.

92. A kit according to any one of claims 81 to 91, wherein the endonuclease further comprises a nuclear localisation sequence effective in mammalian cells.

93. A kit according to any one of claims 81 to 92 wherein said second vector virion encodes a plurality of guide RNAs, each complementary to a different target sequence.

94. A kit according to claim 93 wherein said target sequences are from the same gene.

95. An AAV vector virion for use in a method of modulating intraocular pressure or production of aqueous humour, wherein said vector virion is of serotype ShH10 and comprises a nucleic acid sequence encoding an RNA-guided endonuclease, and is for administration in conjunction with a second AAV vector virion of serotype ShH10, said second vector virion comprising a nucleic acid sequence encoding a guide RNA complementary to a target sequence from an aquaporin gene or a carbonic anhydrase gene and capable of directing said RNA-guided endonuclease to said target sequence.

96. An AAV vector virion for use in a method of modulating intraocular pressure or production of aqueous humour, wherein said vector virion is of serotype ShH10 and comprises a nucleic acid sequence encoding a guide RNA complementary to a target sequence from an aquaporin gene or a carbonic anhydrase gene and is capable of directing an RNA-guided endonuclease to said target sequence; and is for administration in conjunction with a second AAV vector virion of serotype ShH10, said second vector virion comprising a nucleic acid sequence encoding said RNA-guided endonuclease.

97. An AAV vector virion for use according to claim 95 or claim 96 wherein said use is use in the treatment of ocular hypertension and/or glaucoma.

98. An AAV vector virion for use according to claim 97 wherein said glaucoma is primary or secondary glaucoma.

99. An AAV vector virion for use according to claim 97 or claim 98 wherein said primary glaucoma is open-angle glaucoma, closed-angle glaucoma or normal tension glaucoma (NTG).

100. An AAV vector virion for use according to any one of claims 95 to 99, wherein the aquaporin (AQP) gene is AQP1, AQP2, AQP3, AQP4, AQP5, AQP6, AQP7 or AQP11.

101. An AAV vector virion for use according to claim 100, wherein the aquaporin gene is AQP1, AQP4 or AQP5.

102. An AAV vector virion for use according to any one of claims 95 to 99, wherein the carbonic anhydrase (CAR) gene is CAR2, CAR3, CAR4, CR5b, CAR6, CAR8, CAR9, CAR10, CAR12 or CAR14.

103. An MV vector virion for use according to claim 102, wherein the CAR gene is CAR2, CAR3, CAR4, CAR12 or CAR14.

104. An MV vector virion for use in a method of modulating intracranial pressure or production of CSF, wherein said vector virion is of serotype ShH10 and comprises a nucleic acid sequence encoding an RNA-guided endonuclease, and is for administration in conjunction with a second AAV vector virion of serotype ShH10, said second vector virion comprising a nucleic acid sequence encoding a guide RNA complementary to a target sequence from an aquaporin gene or a carbonic anhydrase gene and capable of directing said RNA-guided endonuclease to said target sequence.

105. An MV vector virion for use in a method of modulating intracranial pressure or production of CSF, wherein said vector virion is of serotype ShH10 and comprises a nucleic acid sequence encoding a guide RNA complementary to a target sequence from an aquaporin gene or a carbonic anhydrase gene and is capable of directing an RNA-guided endonuclease to said target sequence; and is for administration in conjunction with a second AAV vector virion of serotype ShH10, said second vector virion comprising a nucleic acid sequence encoding said RNA-guided endonuclease.

106. An AAV vector virion for use according to claim 104 or claim 105 wherein said use is use in the treatment of hydrocephalus or idiopathic intracranial hypertension.

107. An AAV vector virion for use according to claim 106 wherein said hydrocephalus is communicating hydrocephalus or non-communicating hydrocephalus.

108. An AAV vector virion for use according to claim 106 or claim 107 wherein said hydrocephalus is normal pressure hydrocephalus.

109. An AAV vector virion for use according to any one of claims 106 to 108 wherein said hydrocephalus is congenital or acquired.

110. An AAV vector virion for use according to any of claims 104 to 109, wherein the aquaporin (AQP) gene is AQP1, AQP2, AQP3, AQP4, AQP5, AQP6, AQP7 or AQP11.

111. An AAV vector virion for use according to claim 110, wherein the aquaporin gene is AQP1 or AQP4.

112. An MV vector virion for use according to any of claims 104 to 109, wherein the carbonic anhydrase (CAR) gene is CAR2, CAR3, CAR4, CR5b, CAR6, CAR8, CAR9, CAR10, CAR12 or CAR14.

113. An AAV vector virion for use according to claim 112, wherein the CAR gene is CAR2, CAR3, CAR4, CAR12 or CAR14.

114. An AAV vector virion for use according to any one of claims 95 to 113, wherein the RNA-guided endonuclease is a Cas9 enzyme.

115. An AAV vector virion for use according to claim 114, wherein the Cas9 enzyme is Staphylococcus aureus Cas9 (SaCas9), Streptococcus pyogenes Cas9 (SpCas9), Neisseria meningitidis Cas9 (NM Cas9), Streptococcus thermophilus Cas9 (ST Cas9), Treponema denticola Cas9 (TD Cas9), or a variant thereof.

116. A kit according to claim 115, wherein the variant is SpCas9 D1135E, SpCas9 VRER, SpCas9 EQR or SpCas9 VQR.

117. A kit according to any one of claims 95 to 116, wherein the or each RNA-guided endonuclease is catalytically active.

118. A kit according to any one of claims 95 to 116, wherein the or each RNA-guided endonuclease is catalytically dead and further comprises a transcriptional repressor domain.

119. A kit according to claim 118, wherein the transcriptional repressor domain is a Kruppel associated box (KRAB) domain, CS domain, WRPW domain, MXI1, mSin3 interacting domain, or histone demethylase LSD1 domain.

120. A kit according to any one of claims 95 to 119, wherein the endonuclease further comprises a nuclear localisation sequence effective in mammalian cells.

121. A kit according to any one of claims 95 to 120 wherein the nucleic acid sequence encoding a guide RNA encodes a plurality of guide RNAs, each complementary to a different target sequence.

122. A kit according to claim 121 wherein said target sequences are from the same gene.
